(19) United States
(12) Patent Application Publication
(10) Pub. No.: US 2007/0003555 A1
(43) Pub. Date: Jan. 4, 2007

(54) Heat shock protein binding fragments of CD91, and uses thereof

(75) Inventor: Kenneth LeClair, Needham, MA (US)

Correspondence Address: JONES DAY, 222 EAST 41ST ST, NEW YORK, NY, 10017, US

(73) Assignees: UNIVERSITY OF CONNECTICUT HEALTH CENTER, Farmington, CT (US); ANTIGENICS, INC., NewYork, NY (US)

(21) Appl. No.: 10/530,393

(22) PCT Filed: Oct. 7, 2003
(86) PCT No.: PCT/US03/32167

§ 371 Date: Jan. 20, 2006

Related U.S. Application Data
(60) Provisional application No. 60/416,821, filed on Oct. 7, 2002.

(51) Int. Cl. A61K 39/395; G01N 33/574; C07H 21/04; C12P 21/06; C07K 14/82; C07K 16/30; C07K 16/42

(52) U.S. Cl. 424/155.1; 530/350; 435/69.1; 435/320.1; 435/325; 536/23.2; 530/387.2; 530/388.8; 435/7.23

(57) ABSTRACT

The present invention relates to compositions and methods for the use of natural and recombinant p95 forms and fragments as heat shock protein binding proteins. The invention is based, in part, on the Applicant's discovery that a p95 can be recombinantly expressed. The present invention also relates to CD91 polypeptide fragments that comprise at least p95 and additional contiguous sequence from domain II, III, and IV of CD91. The present invention provides nucleic acid molecules encoding a CD91 polypeptide fragment or an analog, derivative or mimetic thereof, CD91 polypeptide fragments, or analogs, derivatives or mimetics thereof, vectors comprising a nucleic acid molecule encoding a CD91 polypeptide fragment, expression vectors comprising a nucleic acid molecule encoding a CD91 polypeptide fragment, eukaryotic and prokaryotic cells recombinantly expressing a CD91 polypeptide fragment, methods of identifying compounds that interact with a CD91 polypeptide fragment or the interaction of a CD91 polypeptide fragment and CD91 ligands, methods for modulating an immune response with the compositions and methods of the invention, and methods for treatment using the compositions and methods disclosed herein.

### 1. INTRODUCTION

The present invention relates to compositions and methods for use of a CD91 polypeptide fragments that bind to heat shock proteins. Specifically, the present invention relates to compositions and methods for use of CD91 heat shock protein-binding fragments that comprise amino acid residues of specific domains of CD91. The invention encompasses nucleic acid molecules, CD91 polypeptide fragments, cells that express CD91 fragments, and antibodies and other molecules that bind such CD91 polypeptide fragments and CD91 polypeptide fragment-HSP complexes. The invention also relates to screening assays to identify compounds that modulate the interaction of an HSP with the CD91 polypeptide fragment, and methods for using such compounds, and methods for using compositions comprising the CD91 polypeptide fragments for the diagnosis and treatment of immune disorders, proliferative disorders, and infectious diseases.

### 2. BACKGROUND OF THE INVENTION

2.1 Heat Shock Proteins

Heat shock proteins (HSPs), also referred to as stress proteins, were first identified as proteins synthesized by cells in response to heat shock. HSPs have been classified into five families, based on molecular weight, HSP100, HSP90, HSP70, HSP60, and sHSP. Many members of these families were found subsequently to be induced in response to other stressful stimuli including nutrient deprivation, metabolic disruption, oxygen radicals, and infection with intracellular pathogens (see Welch, 1993, Sci. Am. 268:56-64; Young, 1990, Annu. Rev. Immunol. 8:401-420; Craig, 1993, Science 260:1902-1903; Gething et al., 1992, Nature 355:33-45; and Lindquist et al., 1988, Annu. Rev. Genetics 22:631-677).

Heat shock proteins are among the most highly conserved proteins in existence. For example, DnaK, the HSP70 from E. coli has about 50% amino acid sequence identity with HSP70 proteins from excoriates (Bardwell et al., 1984, Proc. Natl. Acad. Sci. 81:848-852). The HSP60 and HSP90 families also show similarly high levels of intra-family conservation (Hickey et al., 1989, Mol. Cell Biol. 9:2615-2626; Jindal, 1989, Mol. Cell Biol. 9:2279-2283). In addition, it has been discovered that the HSP60, HSP70 and HSP90 families also comprise proteins that are related to the stress proteins in sequence, for example, having greater than 35% amino acid identity, but whose expression levels are not altered by stress.

Studies on the cellular response to heat shock and other physiological stresses revealed that the HSPs are involved not only in cellular protection against these adverse conditions, but also in essential biochemical and immunological processes in unstressed cells. HSPs accomplish different kinds of chaperoning functions. For example, members of the HSP70 family, located in the cell cytoplasm, nucleus, mitochondria, or endoplasmic reticulum (Lindquist et al., 1988, Ann. Rev. Genetics 22:631-677), are involved in the presentation of antigens to the cells of the immune system, and are also involved in the transfer, folding and assembly of proteins in normal cells. HSPs are capable of binding proteins or peptides, and releasing the bound proteins or peptides in the presence of adenosine triphosphate (ATP) or low pH.

2.2 Immunogenicity of HSP-Peptide Complexes

Srivastava et al. demonstrated immune response to methylcholanthrene-induced sarcomas of inbred mice (1988, Immunol. Today 9:78-83). In these studies, it was found that the molecules responsible for the individually distinct immunogenicity of these tumors were glycoproteins of 96 kDa (gp96) and intracellular proteins of 84 to 86 kDa (p84/p86) (Srivastava et al., 1986, Proc. Natl. Acad. Sci. USA 83:3407-3411; Ullrich et al., 1986, Proc. Natl. Acad. Sci. USA 83:3121-3125). Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava et al., 1988, Immunogenetics 28:205-207; Srivastava et al., 1991, Curr. Top. Microbiol. Immunol. 167:109-123). Further, HSP70 was shown to elicit immunity to the tumor from which it was isolated but not to antigenically distinct tumors. However, HSP70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity to the antigenic peptides (Srivastava, 1993, Adv. Cancer Res. 62:153-177; Udono et al., 1994, J. Immunol., 152:5398-5403; Suto et al., 1995, Science, 269:1585-1588).

Noncovalent complexes of HSPs and peptide, purified from cancer cells, can be used for the treatment and prevention of cancer and have been described in PCT publications WO 96/10411, dated Apr. 11, 1996, and WO 97/10001, dated Mar. 20, 1997 (see also U.S. Pat. No. 5,985,270 issued Nov. 16, 1999).

2.3 CD91

CD91, also known as the alpha (2) macroglobulin (“α2M”) receptor, was initially identified as a protein related to the Low Density Lipoprotein (LDL) receptor and is also referred to as LDL Receptor Related Protein (LRP) (Strickland et al., 1990, J. Biol. Chem. 265:17401-17404; Kristensen et al., 1990, FEBS Lett. 276:151-155). The protein consists of an 420 kDa α subunit, an 85 kDa β subunit and a 39 kDa tightly associated molecule (RAP). The a and β subunits are encoded by a single transcript of approximately 15 Kb in size (Van Leuven et al., 1993, Biochim. Biophys. Acta. 1173:71-74). The receptor has been shown to be present in cells of the monocytic lineage and in hepatocytes, fibroblasts and keratinocytes. CD91 has been shown previously to bind the activated form of the plasma glycoprotein α2M, which binds to and inhibits a wide variety of endoproteinases. CD91 also binds to other ligands such as transforming growth factor β (O'Connor-McCourt et al., 1987, J. Biol. Chem. 262:14090-14099), platelet-derived growth factor (Huang et al., 1984, Proc. Natl. Acad. Sci. U.S.A. 81:342-346), and fibroblast growth factor (Dennis et al., 1989, J. Biol. Chem. 264:7210-7216). The α2M receptor plays a role in endocytosis of a diversity of ligands. In addition to α2M, other ligands of CD91 include lipoprotein complexes, lactoferrin, tissue-type plasminogen activator (tPA), Apolipoprotein E (ApoE), urokinase-type plasminogen activator (uPA), and exotoxins. Thus, CD91 plays roles in a variety of cellular processes, including endocytosis, antigen presentation, cholesterol regulation, ApoE-containing lipoprotein clearance, and chylomicron remnant removal. α2M is thus believed to regulate, and specifically decrease, the activities of its various ligands. Complexed with these various ligands, α2M binds CD91 on the cell surface and is internalized through receptor-mediated endocytosis. Uptake of α2M-complexed ligands has been assumed thus far to be the primary function of CD91, although a role for it in lipid metabolism is also assumed. CD91 ligands other than α2M, such as tissue-specific plasminogen activator-inhibitor complex (Orth et al., 1992, Proc. Natl. Acad. Sci. U.S.A. 89:7422-7426) and urokinase-PAI1 complex (Nykjaer et al., 1992, J. Biol. Chem. 267:14543-14546), have been identified. These ligands attest to a role for CD91 in clearing a range of extracellular, plasma products. Ligands such as lipoprotein complexes, lactoferrin, tissue-type plasminogen activator (tPA), urokinase-type plasminogen activator (uPA), and exotoxins are typically endocytosed by cell upon binding to CD91.

CD91 (herein referred to interchangeably as the alpha (2) macroglobulin receptor, “α2MR”, “the α2M receptor”, or LDL (low-density lipoprotein) receptor-Related Protein (“LRP”), is primarily expressed in liver, brain and placenta. The extracellular domain of the human receptor comprises six 50-amino acid EGF repeats and 31 complement repeats of approximately 40-42 amino acids. The complement repeats are organized, from the amino to the carboxy-terminus, into clusters of 2, 8, 10 and 11 repeats, called Cluster I, II, III and IV (Herz et al., 1988, EMBO J. 7:4119-4127). Cluster I comprises the nucleotide sequence of the p80 fragment, shown to bind heat shock proteins (Binder et al, 2000, Nature Immunology, 1:151-155). Another study points to Cluster II (C1-II), which contains complement repeats 3-10 (CR3-10), as the major ligand binding portion of the receptor (Horn et al., 1997, J. Biol. Chem. 272:13608-13613).

The numerous ligands of CD91 bind to and or interact with the known domains and regions of the receptor molecule as shown in FIG. 4. These ligands include HSPs, which bind to cluster domain region I, ApoE, α2M, tPA, Prourokinase, tPA:PAI-1, uPA:PAI-1, TFPI, Lactopherrin, LPL, and Factor VII which bind or interact with cluster domain II region. ApoE also binds or interacts with cluster domain III region. ApoE, α2M, tPA, Prourokinase, tPA:PAI-1, uPA:PAI-1, Tissue factor pathway inhibitor (TFPI), Lactopherrin, LPL, Factor VIII, and Pseudomonas Exotoxin A all bind or interact with region IV. Additional ligands that are known to bind or interact with CD91 include hepatic lipase, factor Ixa, factor VIIa, MMP-13, MMP-9, shingolipid activator protein (SAP), pregnancy zone protein, complement C3, C1 inhibitor, antithrombin III, heparin cofactor II, alpha1-antitrypsin, thrombospondin-1, thrombospondin-2, rhinovirus, and HIV-Tat.

Human α2M is synthesized as a 1474 amino acid precursor, the first 23 of which function as a signal sequence that is cleaved to yield a 1451 amino acid mature protein (Kan et al., 1985, Proc. Natl. Acad. Sci. U.S.A. 82:2282-2286). In experiments with recombinant protein, the carboxy-terminal 138 amino acids of α2M (representing amino acids 1314-1451 of the mature protein) was found to bind the receptor. This domain has been called the RBD (receptor-binding domain; Salvesent et al., 1992, FEBS Lett. 313:198-202; Holtet et al., 1994, FEBS Lett. 344:242-246). An RBD variant (RBDv), a proteolytic fragment of α2M comprising an additional 15 amino terminal residues (representing amino acids 1314-1451 of the mature protein) binds to the receptor with almost the same affinity as α2M-proteinase (Holtet et al., 1994, FEBS Lett. 344:242-246).

Alignment of α2MR ligands identifies a conserved domain present in the RBDs of α macroglobulins. The conserved sequence spans amino acids 1366-1392 of human α2M. Conserved residues within this domain are Phe₁₃₆₆, Leu₁₃₆₉, Lys₁₃₇₀, Val₁₃₇₃, Lys₁₃₇₄, Glu₁₃₇₇, Val₁₃₈₂, Arg₁₃₈₄ (Nielsen et al., 1996, J. Biol. Chem. 271:12909-12912). Of these, Lys₁₃₇₀ and Lys₁₃₇₄ were shown to be critical for receptor binding (Nielsen et al., 1996, J. Biol. Chem. 271:12909-12912).

Binding of ligands, including the binding to α2M, to CD91 is inhibited by CD91-associated protein (RAP). RAP is a 39 kDa folding chaperone that resides in the endoplasmic reticulum and is required for the normal processing of CD91. RAP has the ability to competitively inhibit the binding of CD91 to all CD91 ligands tested. One study shows RAP to bind to complement repeats C5-C7 in cluster II (C1-II) of CD91 (Horn et al., 1997, J. Biol. Chem. 272:13608-13613); another shows RAP to bind to all two-repeat modules in C1-II except the C9-C10 module (Andersen et al., J. Biol. Chem., Mar. 24, 2000, PMID: 10747921; published electronically ahead of print). Three structural domains, 1, 2 and 3, have been identified in RAP, consisting of amino acid residues 18-112, 113-218 and 219-323, respectively. Ligand competition titration of recombinant RAP domains indicates that determinants for the inhibition of test ligands reside in the C-terminal regions of domains 1 and 3 (Ellgaard et al., 1997, Eur. J. Biochem. 244:544-51).

2.4 Antigen Presentation

Major histocompatibility complex (MHC) molecules present antigens on the cell surface of antigen-presenting cells. Cytotoxic T lymphocytes (CTLs) then recognize MHC molecules and their associated peptides and kill the target cell. Antigens are processed by two distinct antigen processing routes depending upon whether their origin is intracellular or extracellular. Intracellular or endogenous protein antigens, i.e., antigens synthesized within the antigen-presenting cell, are presented by MHC class I (MHC I) molecules to CD8+ cytotoxic T lymphocytes. On the other hand, extracellular or exogenously synthesized antigenic determinants are presented on the cell surface of “specialized” or “professional” antigen presenting cells (APCs) (macrophages, for example) by MHC class II molecules to CD4+ T cells (see, generally, Fundamental Immunology, W.E. Paul (ed.), New York: Raven Press, 1984). This compartmental segregation of antigen processing routes is important to prevent tissue destruction that could otherwise occur during an immune response as a result of shedding of neighboring cell MHC I antigens.

The heat shock protein gp96 chaperones a wide array of peptides, depending upon the source from which gp96 is isolated (for review, see Srivastava et al., 1998, Immunity 8: 657-665; Srivastava, 2002, Nat Rev Immunol. 2(3):185-94.). Tumor-derived gp96 carries tumor-antigenic peptides (Ishii et al., 1999, J. Immunology 162:1303-1309); gp96 preparations from virus-infected cells carry viral epitopes (Suto and Srivastava, 1995, Science 269:1585-1588; Nieland et al., 1998, Proc. Natl. Acad. Sci. USA 95:1800-1805), and gp96 preparations from cells transfected with model antigens such as ovalbumin or β-galactosidase are associated with the corresponding epitopes (Arnold et al., 1995, J. Exp. Med. 182:885-889; Breloer et al., 1998, Eur. J. Immunol. 28:1016-1021). The association of gp96 with peptides occurs in vivo (Menoret and Srivastava, 1999, Biochem. Biophys. Research Commun. 262:813-818). Gp96-peptide complexes, whether isolated from cells (Tamura et al., 1997, Science 278:117-120), or reconstituted in vitro (Blachere et al., 1997, J. Exp. Med. 186:1183-1406) are excellent immunogens and have been used extensively to elicit CD8+ T cell responses specific for the gp96-chaperoned antigenic peptides.

The capacity of gp96-peptide complexes to elicit an immune response is dependent upon the transfer of the peptide to MHC class I molecules of antigen-presenting cells (Suto and Srivastava, 1995, supra). Endogenously synthesized antigens chaperoned by gp96 in the endoplasmic reticulum [ER] can prime antigen-specific CD8+ T cells (or MHC I-restricted CTLs) in vivo; this priming of CD8+ T cells requires macrophages. However, the process whereby exogenously introduced gp96-peptide complexes elicit the antigen-specific CD8+ T cell response is not completely understood since there is no established pathway for the translocation of extracellular antigens into the class I presentation machinery. Yet antigenic peptides of extracellular origin associated with HSPs are somehow salvaged by macrophages, channeled into the endogenous pathway, and presented by MHC I molecules to be recognized by CD8+ lymphocytes (Suto and Srivastava, 1995, supra; Blachere et al., 1997, J. Exp. Med. 186:1315-22).

2.5 HSP-CD91 Interactions

The studies reported by Basu et al. indicate that the heat shock proteins gp96, hsp90, hsp70, and calreticulin are additional ligands for the CD91 (Basu et al., 2001, Immunity 14(3):303-13). Gp96 engages a region of CD91, located in an amino terminal fragment termed the p80 fragment (Binder et al, 2000, Nature immunology, 1:151-155; WO 01/92474). The human gp96-coding gene has been mapped previously by us at chromosome 12 (q24.2 q24.3) (Maki et al., 1993, Somatic Cell Mol. Gen. 19:73-81). It is of interest in this regard that the CD91 gene has been mapped to the same chromosome and at a not too distant location (q13 q14) (Hilliker et al. Genomics 13:472-474). Gp96 appears to bind CD91 directly and not through other ligands such as α2M. Homogenous preparations of gp96, in solution, or cross-linked to a solid matrix, bind to CD91. Indeed, the major ligand for the CD91, α2M, actually inhibits interaction of gp96 with CD91, instead of promoting it, providing evidence that gp96 is a direct ligand for CD91. The 80 kDa protein, p80, shown to bind gp96 is clearly an amino terminal degradation product of the α subunit of CD91 (Binder et al, 2000, Nature Immunology, 1:151-155). Degradation products of CD91 in this size range have also been observed in previous studies (Jensen et al., 1989, Biochem. Arch. 5:171-176), and may indicate the existence of a discrete ectodomain in CD91 which may be particularly sensitive to proteolytic cleavage.

The observations of Basu et al. that α2 macroglobulin and anti-CD91 antibodies inhibit re-presentation by each of the four HSPs completely, indicate that CD91 is the only principle receptor involved in uptake of the four HSPs (Basu et al., 2001, supra). Considering the increasingly obvious role which the HSPs play in innate (Basu et al., 2000, Int. Immunol. 12(11):1539-1546) and adaptive immune response, this observation is somewhat counter-intuitive. However, the data on complete inhibition by two independent means are quite compelling (PCT publication WO 01/92474, dated Dec. 6, 2001). Binder reported significant differences between hsp70 and hsp90/gp96 in their ability to compete for binding to gp96 receptors (Binder et al., 2000, J. Immunol. 165:2582-2587). Another group has also observed similar differences between gp96 and hsp70 (Arnold-Schild et al., 1999, J. Immunol. 162:3757-3760). These differences are not inconsistent with Basu's report pointing to a single receptor for the four HSPs. They simply suggest that the various HSPs interact with a single receptor with widely differing affinities

As shown in Binder et al., the heat shock protein-CD91 interaction provides a new type of function for CD91, or a fragment thereof, a function of a sensor, not only of the extracellular environment with its previously known plasma-based ligands, but also a sensor of the intracellular milieu as well. HSPs such as gp96 are obligate intracellular molecules and are released into the extracellular milieu only under conditions of necrotic (but not apoptotic) cell death (PCT publication WO 01/92474, dated Dec. 6, 2001). Thus, the CD91 may act as a sensor for necrotic cell death, just as the scavenger receptor CD36 and the recently identified phosphatidyl serine-binding protein act as sensors of apoptotic cell death and receptors for apoptotic cells (Savill et al., 1992, J. Clin. Invest. 90:1513-1522; Fadok et al., 2000, Nature 405:85-90). Interaction of the macrophages with the apoptotic cells leads to a down-regulation of the inflammatory cytokines such as TNF (Fadok et al., 2000, supra), while gp96-APC interaction leads to re-presentation of gp96-chaperoned peptides by MHC I molecules of the APC, followed by stimulation of antigen-specific T cells (Suto and Srivastava, 1995, supra) and, in addition, secretion of pro-inflammatory cytokines such as TNF, GM-CSF and IL-12. Interestingly, α2M, an independent ligand for the CD91, inhibits representation of gp96-chaperoned peptides by macrophages. This observation of Binder suggests that re-presentation of gp96-chaperoned peptides can not occur physiologically in blood, but only within tissues as a result of localized necrotic cell death. This is consistent with the complete absence of gp96 or other HSPs in blood under all conditions tested. Together, Binder's observations point towards a possible mechanism whereby the release of HSPs in the blood as a result of severe tissue injury and lysis will not lead to a systemic and lethal pro-inflammatory cytokine cascade.

It is possible, therefore, that CD91 renders it possible for the APCs to sample (i) the extracellular milieu of the blood through α2M and other plasma ligands and (ii) the intracellular milieu of the tissues through HSPs, particularly of the gp96 family. The former permits APCs to implement their primordial phagocytic function, while the latter allows them to execute its innate and adaptive immunological functions. Viewed in another perspective, recognition of apoptotic cells by APCs through CD36 or phophatidyl serine, leads to anti-inflammatory signals, while interaction of the APC with necrotic cells through CD91 leads to pro-inflammatory innate and adaptive immune responses (see Srivastava et al., 1998, Immunity 8: 657-665).

Citation or discussion of a reference herein shall not be construed as an admission that such is prior art to the present invention.

### 3. SUMMARY OF THE INVENTION

The present invention relates natural and recombinant fragments of CD91 which bind HSPs, compositions and methods for the use of such CD91 fragments, and their use as heat shock protein-binding proteins. The invention is based, in part, on the Applicant's discovery of fragments of CD91 that bind HSPs and can be recombinantly expressed and secreted from a cell. In particular, the Applicant has shown that an epitope-tagged p95 and p110 fragments of CD91, as well as other CD91 polypeptides, can be expressed recombinantly in eukaryotic and prokaryotic cells. The present invention also relates to compositions and methods for the use of a CD91 polypeptide fragment that comprises domain I of CD91 and additional contiguous sequence of domain II, natural and recombinant polypeptide forms and fragments, and their use as heat shock protein-binding proteins.

The present invention encompasses CD91 fragment proteins and derivatives thereof that are capable of binding HSPs, herein termed “a CD91 polypeptide fragment of the invention.” The present invention also provides nucleic acid molecules encoding a CD91 polypeptide fragment of the invention or derivative thereof. The invention also encompasses a vector comprising a nucleic acid molecule encoding a CD91 polypeptide fragment, expression vectors, and eukaryotic and prokaryotic cells recombinantly expressing a CD91 polypeptide fragment of the invention. The present invention also provides a method for making a CD91 polypeptide fragment.

In one embodiment, the invention provides a nucleic acid molecule comprising the nucleotide sequence of SEQ ID NO:1, wherein the nucleotide sequence is not flanked by sequences adjacent to SEQ ID NO:1 in the native CD91 nucleotide sequence.

In another embodiment, the invention provides a nucleic acid molecule comprising a nucleotide sequence which encodes a polypeptide consisting of the amino acid sequence of SEQ ID NO:2, 3, 6, 7, 8, 9, 10, 11, or 12.

The invention further provides a nucleic acid molecule comprising a nucleotide sequence which 1) hybridizes over its full length to the nucleotide sequence of SEQ ID NO:1 under conditions of stringent washing and 2) encodes a polypeptide that is capable of (i) being recombinantly produced and secreted into culture medium and (ii) binding to a heat shock protein.

The invention still further provides a nucleic acid molecule comprising a nucleotide sequence which 1) hybridizes over its full length to the nucleic acid sequence of SEQ ID NO:1, 14, 15, 16, or 17 under conditions of stringent washing; and 2) encodes a polypeptide that is capable of (i) being recombinantly produced and secreted into culture medium and (ii) binding to a heat shock protein.

The invention still further provides a nucleic acid molecule comprising a nucleotide sequence which encodes a polypeptide comprising the amino acid sequence of SEQ ID NO:2, 3, 6, 7, 8, 9, 10, 11, or 12, wherein said polypeptide interacts with a heat shock protein. In one embodiment, the isolated nucleic acid molecule of the invention, further comprises a heterologous nucleotide sequence.

The invention still further provides a vector comprising the nucleic acid molecule of the invention, as described herein above.

In one aspect, the invention provides an expression vector comprising the nucleic acid molecule of the invention, wherein the nucleotide sequence is operatively associated with a nucleotide regulatory sequence that controls expression of the nucleotide sequence in a host cell.

The invention further provides a genetically engineered host cell comprising the nucleic acid molecule of the invention, wherein the nucleotide sequence is operatively associated with a nucleotide regulatory sequence that controls expression of the nucleotide sequence in the host cell. The invention still further provides an host cell comprising the expression vector of the invention as described herein above.

In another embodiment, the invention provides a polypeptide comprising the amino acid sequence of SEQ ID NO:2, 3, 6, 7, 8, 9, 10, 11, or 12, wherein the amino acid sequence is not flanked by sequences which are adjacent to SEQ ID NO:2, 3, 6, 7, 8, 9, 10, 11, or 12, respectively, in the native CD91 polypeptide sequence.

The invention also provides an isolated polypeptide comprising a contiguous amino acid sequence, wherein said amino acid sequence consists of amino acid residues 1-851 of SEQ ID NO:11, fused to one or more contiguous amino acids of amino acid residues 852-4420 of SEQ ID NO:11.

The invention further provides an isolated polypeptide encoded by a nucleic acid molecule that hybridizes under stringent conditions to a complement of SEQ ID NO: 1, 14, 15, 16, or 17, wherein said polypeptide binds to a heat shock protein.

The invention still further provides an isolated polypeptide encoded by a nucleic acid molecule which hybridizes under stringent conditions to a complement of a nucleic acid molecule consisting of a nucleotide sequence that encodes a polypeptide consisting of the amino acid sequence of SEQ ID NO:2, 6, 8, 10, 11, or 12, wherein said polypeptide binds to a heat shock protein. In certain embodiments, the heat shock protein is selected from the group consisting of a gp96, hsp 90, hsp 70, and calreticulin. In one embodiment, the heat shock protein is gp96.

The invention also provides a fusion polypeptide comprising the amino acid sequence of SEQ ID NO:2, 3, 6, 7, 8, 9, 10, 11, or 12 and a heterologous amino acid sequence.

The invention also provides a kit comprising the CD91 polypeptide fragment of the invention in one or more containers. In one embodiment, a kit is provided, packaged in one or more containers, comprising a CD91 polypeptide fragment of the invention, nucleic acid encoding an CD91 polypeptide fragment of the invention, or a cell expressing a CD91 polypeptide fragment of the invention. In one embodiment, the kit the CD91 polypeptide fragment of the invention, nucleic acid encoding an CD91 polypeptide fragment of the invention, or cell expressing a CD91 polypeptide fragment of the invention is purified. In another embodiment, the kit further comprises instructions for use in treating an autoimmune disorder, an infectious disease, or a proliferative disorder.

According to another aspect of the invention, the invention provides a method for making the polypeptide of the invention, comprising the steps of (a) culturing a cell comprising a recombinant nucleotide sequence encoding the polypeptide of the invention, under conditions such that the polypeptide is expressed by said cell, and (b) recovering the expressed polypeptide from the cell culture.

The invention further provides an antibody or fragment thereof that immunospecifically binds to a CD91 polypeptide fragment that binds an HSP, wherein the CD91 polypeptide fragment comprises the amino acid sequence of SEQ ID NO.:2, 3, 6, 7, 8, 9, 10, 11, or 12. In various embodiments, the antibody is a polyclonal antibody, a monoclonal antibody, a humanized antibody, a single chain antibody, or a chimeric antibody. In another embodiment, the antibody or fragment thereof is a Fab fragment. The invention provides an anti-idiotype antibody which binds to the antibody or fragment of the invention described herein above.

The invention provides a kit comprising in one or more containers the antibody of the invention described herein above, and optionally instructions for its use to detect a CD91 polypeptide fragment.

The invention also provides a method for treating a CD9′-related disease or disorder comprising administering the polypeptide as described herein above to a mammal in need thereof in an amount effective to treat the disease or disorder. In certain embodiments, the disease or disorder is an autoimmune disorder, a disease or disorder involving disruption of antigen presentation or endocytosis, a disease or disorder involving cytokine clearance or inflammation, a proliferative disorder, a viral disorder or other infectious disease, hypercholesterolemia, Alzheimer's disease, diabetes, or osteoporosis.

The invention also provides a method for modulating an immune response comprising administering a CD91 polypeptide fragment of the invention, a compound identified by the methods of the invention, or a complex of an HSP and a CD91 polypeptide fragment of the invention to a mammal in need thereof in an amount effective to modulate an immune response. In certain embodiments, an immune response is stimulated. In certain other embodiments, an immune response is inhibited or blocked.

The invention also provides a method for modulating an immune response comprising administering to a mammal a purified compound identified by the methods of the invention that modulates the interaction of a CD91 ligand (e.g., a heat shock protein) with CD91. In one embodiment, the compound is an agonist which enhances the interaction of a CD91 ligand (e.g., a heat shock protein) with CD91. In another embodiment of this method the compound in an antagonist that interferes with the interaction between a CD91 ligand (e.g., a heat shock protein) with CD91.

In another embodiment, the invention provides a method for identifying a compound that modulates an HSP-CD9′-mediated process, comprising (a) contacting a test compound with a heat shock protein and a CD91 polypeptide fragment that binds a CD91 ligand, and (b) measuring the level of the CD91 polypeptide fragment activity or expression, such that if the level of activity or expression measured in (b) differs from the level of the CD91 polypeptide fragment activity or expression measured in the presence the heat shock protein but in the absence of the test compound, then a compound that modulates an HSP-CD91-mediated process is identified. In a specific embodiment, the compound identified is an antagonist which interferes with the interaction of the heat shock protein with the CD91 polypeptide fragment. In certain embodiments, the test compound is a small molecule or a peptide. In certain embodiments, the peptide comprises at least 5 consecutive amino acids of the CD91 polypeptide fragment. In certain embodiments, the HSP-CD91-mediated process affects an autoimmune disorder, a disease or disorder involving disruption of antigen presentation or endocytosis, a disease or disorder involving cytokine clearance or inflammation, a proliferative disorder, a viral disorder or other infectious disease, hypercholesterolemia, Alzheimer's disease, diabetes, or osteoporosis.

The invention further provides a method for identifying a compound that modulates the binding of a heat shock protein to CD91, comprising (a) contacting a heat shock protein with a CD91 polypeptide fragment, or derivative thereof, which CD91 polypeptide fragment that binds a CD91 ligand, in the presence of a test compound under conditions conducive to binding, and (b) measuring the level of heat shock protein bound to the CD91 polypeptide fragment or derivative thereof, such that if the level of bound heat shock protein measured in (b) differs from the level of heat shock protein measured bound to the CD91 polypeptide fragment or derivative thereof measured under said conditions in the absence of the test compound, then a compound that modulates the binding of an HSP to the CD91 polypeptide fragment is identified. In one specific embodiment, the CD91 polypeptide fragment, or derivative thereof, is immobilized to a solid support. In another specific embodiment, the solid support is a microtiter dish. In another specific embodiment, the level of bound heat shock protein is measured using a heat shock protein-specific antibody. In another specific embodiment, the heat shock protein is labeled and the level of bound heat shock protein is measured by detecting the label. In another specific embodiment, the heat shock protein is labeled with a fluorescent label.

The CD91 polypeptide fragment of the invention is a polypeptide fragment of CD91 that comprises at least domain I (p95) of CD91 (but not full length CD91). In certain embodiments the CD91 polypeptide fragment comprises additional contiguous amino acids extending into domains II, II, or IV or CD91.

In various embodiments, the CD91 polypeptide fragment ligand is gp96 or another heat shock protein.

The term “HSP-CD91 interaction” as used herein refers to a process dependent and/or responsive, either directly or indirectly, to the interaction of HSP with a CD91 protein. Such processes include processes that result from an aberrant level of expression, synthesis and/or activity of a CD91 protein, such as endocytic activities relating to the binding of the various CD91 ligands, including but not limited to HSPs. Such processes can include, but are not limited to, endocytosis, antigen presentation, cholesterol regulation, apoE-containing lipoprotein clearance, and chylomicron remnant removal.

The terms “HSP-CD91 disorder” and “HSP-CD91-related condition”, as used herein, refers to a disorder and a condition, respectively, involving a HSP-CD91 protein interaction. Such disorders and conditions may result, for example, from an aberrant ability of a CD91 protein to interact with HSP, perhaps due to aberrant levels of HSP and/or CD91 protein expression, synthesis and/or activity relative to levels found in normal, unaffected, unimpaired individuals, levels found in clinically normal individuals, and/or levels found in a population whose levels represent a baseline, average HSP and/or CD91 protein levels. Such disorders may include, but are not limited to, autoimmune disorders, diseases and disorders involving disruption of antigen presentation and/or endocytosis, diseases and disorders involving cytokine clearance and/or inflammation, proliferative disorders, viral disorders and other infectious diseases, hypercholesterolemia, Alzheimer's disease, diabetes, and osteoporosis.

The term “CD91 ligand” as used herein, refers to a molecule capable of binding to a CD91 protein or a CD91 polypeptide fragment. Such CD91 ligands include but are not limited to, CD91 protein complexes (complexes of CD91 and another molecule), heat shock proteins, and heat shock protein complexes (complexes of a heat shock protein and another molecule). In addition, CD91 ligands also include molecules which can readily be identified as CD91 ligands using standard binding assays well known in the art. Such CD91 ligands are typically endocytosed by cells upon binding to a CD91 protein.

### 4. BRIEF DESCRIPTION OF THE FIGURES

FIG. 1. Pictorial representation of CD91, a multiligand receptor.

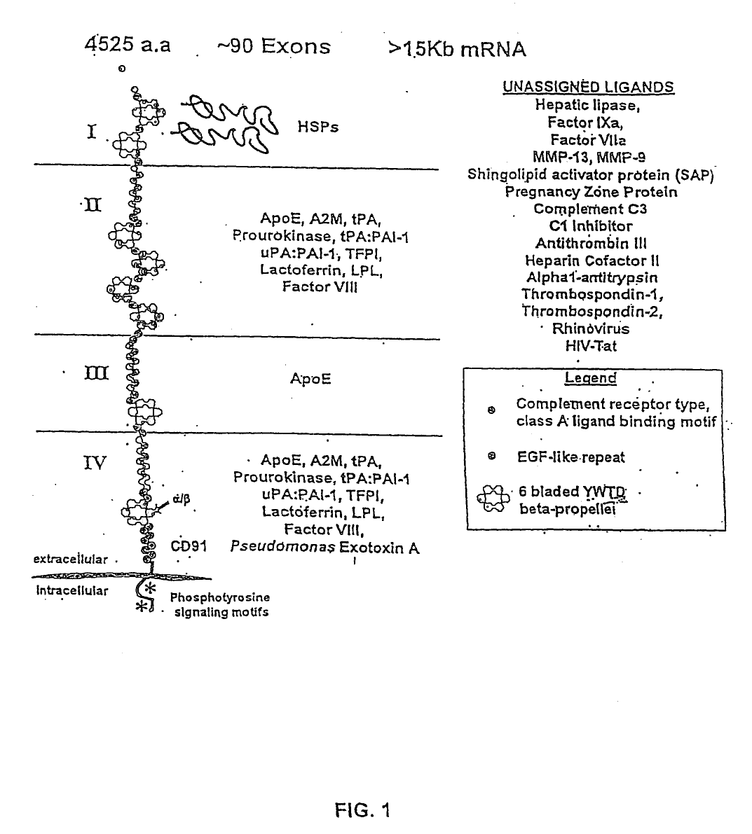

FIG. 2A-C. Expression and purification of recombinant human CD91-p95 Fragment with anti-HA Mab affinity purification. (1A) The p95 epitope-tagged protein eluted from the HA affinity was visualized by staining with Coomassie Brilliant Blue. (1C) anti-CD91 antibody (8G1) Western blot indicating presence of epitope-tagged protein in elution. (1B) The eluate was applied to anti-HA affinity resins for single step purification of recombinant human CD91 protein fragment.

FIG. 3. Mouse CD91-p80 protein structural motifs (SEQ ID NO:4). Residues 25-66 correspond to CR1, residues 70-110 correspond to CR2, residues 111-149 correspond to EGF-like #1 domain, residues 150-189 correspond to EGF-like #2 domain, residues 190-473 correspond to YWTD β-propeller #1 region, residues 474-520 correspond to EGF-like #3 domain, residues 521-799 correspond to YWTD α-propeller #2, and residues 800-843 correspond to EGF-like #4.

FIG. 4. Structural motifs of human CD91 protein (SEQ ID NO:5) GenBank accession no. S02392. Amino acid residues of domains I-IV are indicated with repeats and beta propeller regions identified by residue numbers. A transmembrane domain is identified at amino acid residues 4421-4444 and an intracellular domain at residues 4445-4544. The remaining coding sequence without the transmembrane domains is residues 1-4420 (SEQ ID NO:11).

FIG. 5. Shows a photograph of an acrylamide gel of Ni NTA-purified recombinant CD91 fragments of p95 (first lane) and p110 (second lane).

### 5. DETAILED DESCRIPTION OF THE INVENTION

The present invention relates to compositions and methods for the use of CD91 polypeptide fragments. In particular, the present invention provides a composition comprising CD91 polypeptide fragments comprising the ligand-binding domain, an isolated nucleic acid molecule encoding a CD91 polypeptide fragment, isolated and/or recombinant cells expressing the nucleic acid and protein molecules of the invention, antibodies to CD91 polypeptide fragments, molecules and compounds that modulate the interaction of a CD91 polypeptide fragment with a CD91 ligand, such as an HSP. The invention further encompasses methods for the use of a ligand-binding CD91 polypeptide fragment as a dominant negative inhibitor of the heat shock protein receptor CD91. The invention further encompasses screening assays to identify compounds that modulate the interaction of a CD91 polypeptide fragment with an HSP, or another CD91 ligand, and methods for the use of these molecules and complexes for the diagnosis and treatment of immune disorders, proliferative disorders, and infectious diseases.

The CD91 polypeptide fragment of the invention is a ligand-binding fragment of CD91. The term “CD91 ligand” as used herein, refers to a molecule capable of binding to CD91. Such CD91 ligands include but are not limited to heat shock proteins and heat shock protein complexes. In addition, ligands also include molecules which can readily be identified as CD91 ligands using standard binding assays well known in the art. Such CD91 ligands are typically endocytosed by cell upon binding to CD91.

An HSP useful in the practice of the invention may be selected from among any cellular protein that satisfies the following criteria: the intracellular concentration of an HSP increases when a cell is exposed to a stressful stimulus; an HSP can bind other proteins or peptides, and can release the bound proteins or peptides in the presence of adenosine triphosphate (ATP) or low pH; or an HSP possesses at least 35% homology with any cellular protein having any of the above properties. Preferably, the HSP used in the compositions and methods of the present invention includes, but are not limited to, HSP90, gp96, BiP, HSP70, HSP 110, grp 170, DnaK, Hsc70, caketiculin, or a smallHSP (sHSP), alone or in combination. In a preferred embodiment, an HSP is a mammalian (e.g., mouse, rat, primate, domestic animal such as dog, cat, cow, horse) HSP, and is most preferably, human.

HSPs useful in the practice of the invention include, but are not limited to, members of the HSP60 family, HSP70 family, HSP90 family, HSP110 family, sHSP family, calreticulin, and other proteins in the endoplasmic reticulum that contain thioredoxin-like domain(s), such as, but not limited to, ERp72 and ERp61.

HSP analogs, muteins, derivatives, and fragments can also be used in place of HSPs according to the invention. An HSP peptide-binding “fragment” for use in the invention refers to a polypeptide, comprising a HSP peptide-binding domain that is capable of becoming non-covalently associated with a peptide to form a complex that is capable of modulating a CD91-mediated immune response. In one embodiment, an HSP peptide-binding fragment is a polypeptide comprising an HSP peptide-binding domain of approximately 100 to 200 amino acids.

Databases can also be searched to identify sequences with various degrees of similarities to a query sequence using programs, such as FASTA and BLAST, which rank the similar sequences by alignment scores and statistics. Such nucleotide sequences of non-limiting examples of HSPs that can be used for preparation of the HSPs used in the methods of the invention are as follows: human HSP70, Genbank Accession No. NM_{—}005345, Sargent et al., 1989, Proc. Natl. Acad. Sci. U.S.A., 86:1968-1972; human HSP90, Genbank Accession No. X15183, Yamazaki et al., Nucl. Acids Res. 17:7108; human gp96: Genbank Accession No. X15187, Maki et al., 1990, Proc. Natl. Acad. Sci., 87: 5658-5562; human BiP: Genbank Accession No. M19645; Ting et al., 1988, DNA 7: 275-286; human HSP27, Genbank Accession No. M24743; Hickey et al., 1986, Nucleic Acids Res. 14:4127-45; mouse HSP70: Genbank Accession No. M35021, Hunt et al., 1990, Gene, 87:199-204; mouse gp96: Genbank Accession No. M16370, Srivastava et al., 1987, Proc. Natl. Acad. Sci., 85:3807-3811; and mouse BiP: Genbank Accession No. U16277, Haas et al., 1988, Proc. Natl. Acad. Sci. U.S.A., 85: 2250-2254. Due to the degeneracy of the genetic code, the term “HSP sequence”, as used herein, refers not only to the naturally occurring amino acid and nucleotide sequence but also encompasses all the other degenerate sequences that encode the HSP.

The aforementioned HSP families also contain proteins that are related to HSPs in sequence, for example, having greater than 35% amino acid identity, but whose expression levels are not altered by stress. Therefore, it is contemplated that the definition of heat shock or stress protein, as used herein, embraces other proteins, mutants, analogs, and variants thereof having at least 35% to 55%, preferably 55% to 75%, and most preferably 75%, 85%, 90%, 95%, or 98% amino acid identity with members of these families whose expression levels in a cell are enhanced in response to a stressful stimulus. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The isolated CD91 polypeptide fragments of the invention may include any CD91 fragment containing a ligand binding domain. Such CD91 polypeptide fragments may be capable of modulating, i.e., suppressing or enhancing, an immune response in a mammal.

The CD91 polypeptide fragments for use in the invention can be purified from natural sources, chemically synthesized, or recombinantly produced. The HSPs and/or antigenic molecules for use in the invention can be purified from natural sources, chemically synthesized, or recombinantly produced. Although the HSPs may be allogeneic to the patient, in a preferred embodiment, the HSPs are autologous to the patient to whom they are administered.

5.1 Compositions of the Invention

The present invention provides compositions for use of CD91 polypeptide fragments in modulating the interaction between CD91 and a CD91 ligand, such as, for example, an HSP. Such compositions can be used in methods to modulate an immune response. Such compositions include CD91 polypeptide fragments, nucleic acids that encode CD91 polypeptide fragments, antibodies that specifically recognize HSP-CD91 polypeptide fragment complexes or recognize HSP binding domain of CD91, isolated recombinant or genetically engineered cells that express CD91 polypeptide fragments, HSP-CD91 polypeptide fragment complexes, and isolated and recombinant cells that contain recombinant CD91 polypeptide fragments and/or HSP sequences. In addition, in various methods of the invention, sequences encoding a CD91 polypeptide fragment and an HSP are used for immunotherapy. In other embodiments, CD91 polypeptide fragments of the invention and CD91-HSP complexes are used for immunotherapy. Such compositions can be used, for example, in immunotherapy against proliferative disorders, infectious diseases, and other HSP-CD91-related disorders. Methods for the synthesis and production of such compositions are described herein.

5.1.1 Nucleic Acids of the Invention

A nucleic acid molecule encoding a CD91 polypeptide fragment of the invention includes:

(a) a nucleic acid molecule consisting of the DNA sequence of p95 (SEQ ID NO:1, the human CD91-p95 DNA sequence);

(b) a nucleic acid molecule that encodes a p95 protein product (SEQ ID NOS: 2 and 3);

(c) a nucleic acid molecule that encodes a CD91 fragment protein product that comprises at least amino acid residues 1-851 of SEQ ID NO:5, and can comprise additional contiguous sequence of SEQ ID NO:5 but does not encompass the entire receptor protein;

(d) nucleic acid molecules that encode fusion proteins comprising a CD91 polypeptide fragment of the invention fused to a heterologous polypeptide;

(e) nucleic acid molecules that 1) hybridize over their full length to a second nucleic acid molecule which encodes a polypeptide consisting of the amino acid sequence of SEQ ID NO:1 under conditions of stringent washing; and 2) encode a polypeptide that is capable of being recombinantly produced and released into the culture media and binds to heat sock proteins;

(f) nucleic acid molecules that 1) hybridize over their full length to a second nucleic acid molecule consisting of the nucleotide sequence of SEQ ID NO:2, 6, 8, or 10 under conditions of stringent washing; and 2) encode a polypeptide that is capable of being recombinantly produced and secreted into culture media and binds to heat sock proteins; and

(g) nucleic acid molecules greater than 20, 30, 40, 50, 60, 70, 80, 90, 100, or more base pairs long that have at least 80%, 85%, 90%, 95%, 98%, or more nucleotide sequence identity to the nucleotide sequences of (a)-(f) above, wherein such nucleic acid molecule which encodes a gene product having the capacity to (a) bind a heat shock protein and (b) be recombinantly expressed and released from a cell.

As used herein, a nucleic acid molecule that “hybridizes over its full length” refers to a nucleic acid molecule that hybridizes to a specified nucleic acid molecule which is substantially the same length of the specified nucleic acid molecule, such that the duplexes formed after hybridization do not contain intervening sequences of single stranded regions of DNA, such as introns.

As used herein, a nucleic acid molecule that is “beterologous” to a specified nucleic acid molecule refers to a nucleic acid molecule that is derived from a different organism than the organism from which the specified nucleic acid molecule is derived.

Examples of nucleic acid molecules encoding CD91 polypeptide fragments include, but are not limited to, human CD91-p95 DNA sequence (SEQ ID NO:1); human CD91-p110 DNA sequence (SEQ ID NO:14); human CD91-p282 DNA sequence (SEQ ID NO:15); human CD91-p373 DNA sequence (SEQ ID NO:16); human CD91-p494 DNA sequence (SEQ ID NO:17); and human CD91 fragment that does not include the transmembrane domain (SEQ ID NO:18).

In certain embodiments, the nucleic acid molecules of the invention described above can be isolated.

The nucleic acid molecules of the invention further include nucleotide sequences that encode polypeptides having at least 30%, 35%, 40%, 45%, 50%, 55%, 60%, 65%, 70%, 75%, 80%, 85%, 90%, 95%, 98%, or higher amino acid sequence identity to the polypeptides encoded by the p95 and CD91 polypeptide fragment nucleotide sequences of (a)-(d) above having the capacity to (a) bind a heat shock protein and (b) be recombinantly expressed and released from a cell.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of overlapping positions×100%). In one embodiment, the two sequences are the same length.

The determination of percent identity between two sequences can also be accomplished using a mathematical algorithm as described in Section 5 above.

The percent identity between two sequences can be determined using techniques similar to those described above, with or without allowing gaps. In calculating percent identity, typically only exact matches are counted.

The nucleic acid molecules of the invention further include: (a) any nucleotide sequence that hybridizes to a CD91 fragment nucleic acid molecule of the invention described in (a)-(e) above, under stringent conditions, e.g., hybridization to filter-bound DNA in 6× sodium chloride/sodium citrate (SSC) at about 45° C. followed by one or more washes in 0.2×SSC/0.1% SDS at about 50-65° C., or (b) under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel F. M. et al., eds., 1989, Current Protocols in Molecular Biology, Vol. I, Green Publishing Associates, Inc., and John Wiley & sons, Inc., New York, at pp. 6.3.1-6.3.6 and 2.10.3). Preferably the CD91 fragment nucleic acid molecule that hybridizes under conditions described under (a) and (b), above, is one that comprises the complement of a nucleic acid molecule that encodes a CD91 fragment protein product. In a preferred embodiment, nucleic acid molecules that hybridize under conditions (a) and (b), above, encode protein products, e.g., protein products functionally equivalent to a CD91 fragment protein product. Preferably, the nucleic acids of the invention encode human CD91 protein fragments.

Functionally equivalent CD91 fragment protein products include naturally occurring CD91 polypeptide fragments of CD91 present in the same or different species and variants thereof. Functionally equivalent CD91 fragment protein products also include protein products that retain at least one of the biological activities of a CD91 fragment protein product, and/or which are recognized by and bind to antibodies (polyclonal or monoclonal) directed against a CD91 fragment protein product.

Among the nucleic acid molecules of the invention are deoxyoligonucleotides (“oligos”) which hybridize under highly stringent or stringent conditions to the nucleic acid molecules described above that encode CD91 polypeptide fragments. In general, for probes between 14 and 70 nucleotides in length the melting temperature (Tm) is calculated using the formula: Tm(° C.)=81.5+16.6(log[monovalent cations(molar)])+0.41(% G+C)−(500/N) where N is the length of the probe. If the hybridization is carried out in a solution containing formamide, the melting temperature is calculated using the equation Tm(° C.)=81.5+16.6(log[monovalent cations(molar)])+0.41(% G+C)−(0.61% formamide)−(500/N) where N is the length of the probe. In general, hybridization is carried out at about 20-25 degrees below Tm (for DNA-DNA hybrids) or 10-15 degrees below Tm (for RNA-DNA hybrids).

Exemplary highly stringent conditions may refer, e.g., to washing in 6×SSC/0.05% sodium pyrophosphate at 37° C. (for about 14-base oligos), 48° C. (for about 17-base oligos), 55° C. (for about 20-base oligos), and 60° C. (for about 23-base oligos).

The nucleic acid molecules of the invention further comprise the complements of the nucleic acids described above. Such molecules can, for example, act as antisense molecules, useful, for example, in CD91 nucleic acid regulation, and/or as antisense primers in amplification reactions of CD91 nucleic acid sequences.

Nucleic acid sequences of the invention encoding a CD91 fragment protein product or complements thereof, may be used as part of ribozyme and/or triple helix sequences, also useful for CD91 nucleic acid regulation.

Still further, the nucleic acid molecules of the invention may be used as components of diagnostic methods whereby, for example, the presence of a particular CD91 fragment nucleotide sequence involved in a CD91-related disorder or a HSP-CD91-related disorder, such as a proliferative cell disorder, an autoimune disorder, or an infectious disease is detected.

p95 fragment nucleic acid molecules refer to p95 nucleic acid sequences that can be at least 10, 12, 15, 20, 30, 40, 50, 60, 70, 80, 90, 100, 200, 300, 400, 500, 600, 700, 800, 900, 1000, 1050, or more contiguous nucleotides in length. Alternatively, the fragments can comprise sequences that encode at least 10, 20, 30, 40, 50, 60, 70, 80 or more contiguous amino acid residues of the p95 fragment protein products. In one embodiment, the p95 fragment nucleic acid molecule encodes a gene product exhibiting at least one biological activity of a corresponding CD91 protein product. Fragments can also refer to portions of p95 polypeptide fragment coding regions that encode domains of, or mature p95 fragment protein products.

With respect to CD91 fragment nucleic acid sequence variants or polymorphisms, any and all nucleotide variations and resulting amino acid polymorphisms or variations that are the result of natural allelic variation of the CD91 fragment are intended to be within the scope of the present invention. Allelic variants or polymorphism include, but are not limited to, ones that alter the functional activity of the CD91 fragment protein product.

With respect to the cloning of additional allelic variants of the human CD91 fragment nucleic acid fragment of CD91 and homologous and orthologs from other species, the isolated CD91 fragment nucleic acid sequences disclosed herein may be labeled and used to screen a cDNA library constructed from mRNA obtained from appropriate cells or tissues derived from the organism of interest. The hybridization conditions used should generally be of a lower stringency when the cDNA library is derived from an organism different from the type of organism from which the labeled sequence was derived, and can routinely be determined based on, e.g., relative relatedness of the target and reference organisms.

Alternatively, the labeled fragment may be used to screen a genomic library derived from the organism of interest, again, using appropriately stringent conditions. Appropriate stringency conditions are well known to those of skill in the art as discussed above, and will vary predictably depending on the specific organisms from which the library and the labeled sequences are derived. For guidance regarding such conditions see, for example, Sambrook, et al., 1989, Molecular Cloning, A Laboratory Manual, Second Edition, Cold Spring Harbor Press, N.Y.; and Ausubel, et al., 1989-1999, Current Protocols in Molecular Biology, Green Publishing Associates and Wiley Interscience, N.Y., both of which are incorporated herein by reference in their entirety.

Further, a CD91 fragment nucleic acid molecule of a CD91 allelic gene variant may be isolated from, for example, human nucleic acid, by performing PCR using two degenerate oligonucleotide primer pools designed on the basis of amino acid sequences within the CD91 fragment protein product disclosed herein. The template for the reaction may be cDNA obtained by reverse transcription of mRNA prepared from, for example, human or non-human cell lines or tissue known or suspected to express a wild type or mutant CD91 fragment nucleic acid of a CD91 allele. In one embodiment, the allelic variant is isolated from an individual who has a CD91-mediated disorder or a HSP-CD91 related disorder.

The PCR product may be subcloned and sequenced to ensure that the amplified sequences represent the sequences of a CD91 fragment nucleic acid sequence. The PCR fragment may then be used to isolate a full length cDNA clone by a variety of methods. For example, the amplified fragment may be labeled and used to screen a bacteriophage cDNA library. Alternatively, the labeled fragment may be used to isolate genomic clones via the screening of a genomic library.

A cDNA of an allelic, e.g., mutant, variant of the CD91 fragment nucleic acid may be isolated, for example, by using PCR, a technique that is well known to those of skill in the art. In this case, the first cDNA strand may be synthesized by hybridizing an oligo-dT oligonucleotide to mRNA isolated from tissue known or suspected to be expressed in an individual putatively carrying a mutant CD91 fragment sequence, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known to those of skill in the art. By comparing the DNA sequence of the mutant CD91 fragment sequence to that of the normal CD91 fragment sequence, the mutation(s) responsible for the loss or alteration of function of the mutant CD91 fragment protein product can be ascertained.

Alternatively, a genomic library can be constructed using DNA obtained from an individual suspected of or known to carry a mutant CD91 fragment sequence, or a cDNA library can be constructed using RNA from a tissue known, or suspected, to express a mutant CD91 fragment sequence. An unimpaired CD91 fragment nucleic acid or any suitable fragment thereof may then be labeled and used as a probe to identify the corresponding mutant CD91 fragment sequence in such libraries. Clones containing the mutant CD91 fragment nucleic acid sequences may then be purified and subjected to sequence analysis according to methods well known to those of skill in the art.

Additionally, an expression library can be constructed utilizing cDNA synthesized from, for example, RNA isolated from a tissue known, or suspected, to express a mutant CD91 allele in an individual suspected of or known to carry such a mutant allele. In this manner, gene products made by the putatively mutant tissue may be expressed and screened using standard antibody screening techniques in conjunction with antibodies raised against the normal CD91 gene product (For screening techniques, see, for example, Harlow and Lane, eds., 1988, “Antibodies: A Laboratory Manual”, Cold Spring Harbor Press, Cold Spring Harbor.)

In cases where a mutation in a CD91 fragment sequence results in an expressed protein product with altered function (e.g., as a result of a missense or a frameshift mutation), a polyclonal set of anti-CD91 fragment protein product antibodies are likely to cross-react with the mutant CD91 fragment protein product. Library clones detected via their reaction with such labeled antibodies can be purified and subjected to sequence analysis according to methods well known to those of skill in the art.

CD91 fragment mutations or polymorphisms can further be detected using PCR amplification techniques. Primers can routinely be designed to amplify overlapping regions of a whole CD91 fragment sequence.

The invention also includes nucleic acid molecules, preferably DNA molecules, that are the complements of the nucleotide sequences of the preceding paragraphs.

In certain embodiments, the nucleic acid molecules of the invention are present as part of nucleic acid molecules comprising nucleic acid sequences that contain or encode heterologous (e.g., vector, expression vector, or fusion protein) sequences.

5.1.2 Amino Acids and Polypeptides of the Invention

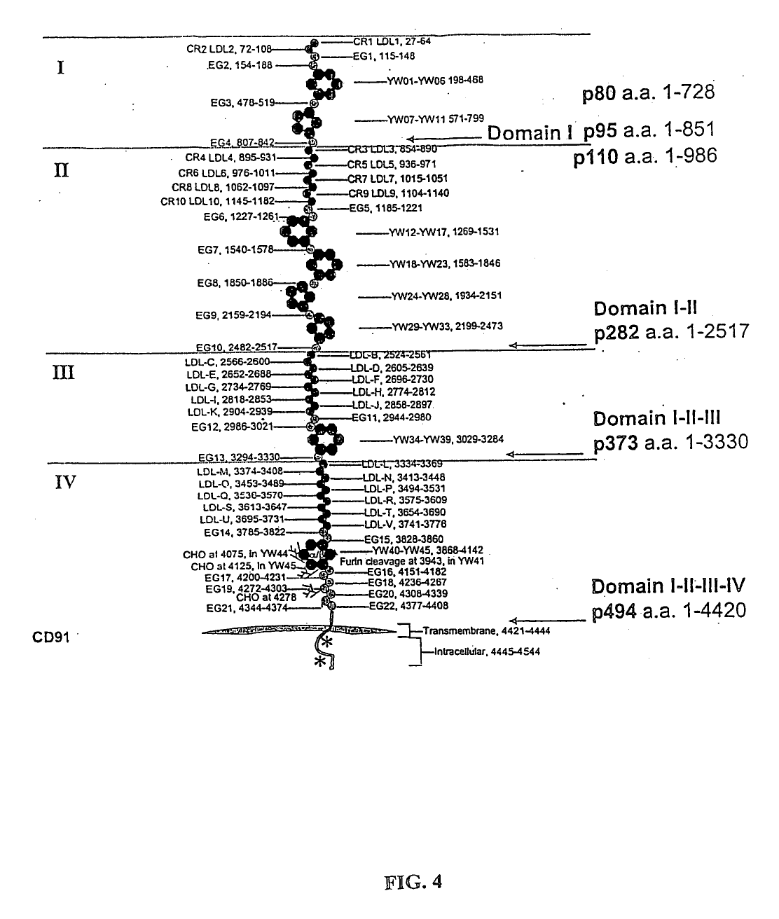

The present invention encompasses CD91 polypeptide fragments and analogs, derivatives and mimetics thereof that are capable of binding HSPs. Examples of CD91 polypeptide fragments include, but are not limited to, human CD91-p95 amino acid sequence (SEQ ID NO:2); soluble recombinant epitope-tagged human CD91-p95 amino acid sequence (SEQ ID NO:3); human CD91-p95 amino acid sequence (SEQ ID NO:13) with residues 1-851 corresponding to domain I and a 5.2 kD tag following amino acid residue 851; human CD91-p282 protein amino acid sequence (SEQ ID NO:6) with amino acid residues 1-851 corresponding to domain I and residues 852-2517 correspond to domain II, and a 5.2 kD tag following amino acid residue 2517; human CD91-p373 protein amino acid sequence (SEQ ID NO:8) with residues 1-851 corresponding to domain I, residues 852-2517 corresponding to domain II, and residues 2518-3330 corresponding to domain III and a 5.2 kD tag following amino acid residue 3330; and human CD91-p494 protein amino acid sequence (SEQ ID NO:10) with residues 1-851 corresponding to domain I, residues 852-2517 correspond to domain II, residues 2518-3330 corresponding to domain III, and residues 3330-4420 corresponding to domain IV and a 5.2 kD tag is shown following amino acid residue 4420. Other examples of CD91 polypeptide fragments include, but are not limited to recombinant CD91-“LRP25”. In one embodiment, the CD91 polypeptide fragment of the invention is not LRP25. Examples of CD91 polypeptide fragments of the invention include, but are not limited to, fragments of the mouse CD91 amino acid sequence (GenBank No. 109553), the human CD91 amino acid sequence (GenBank No. 88083), or the chicken CD91 amino acid sequence (GenBank No. 1079416). Such fragments comprise one or more of the domains or fragments thereof corresponding the CD91 domains shown in FIG. 4. In one embodiment, the CD91 polypeptide fragment of the invention is not a full length CD91. In one embodiment, the CD91 polypeptide fragment of the invention does not comprise the CD91 transmembrane domain, such as, but not limited to the transmembrane domain shown in FIG. 4.

CD91 polypeptide fragments, can be prepared for a variety of uses. For example, such gene products, can be used for the generation of antibodies, in diagnostic assays, or for mapping and the identification of other cellular or extracellular gene products involved in the regulation of a HSP-CD91 related disorders, such as cancer, infectious disease or autoimmune disorders.

A “CD91 polypeptide fragment” of the invention, includes those polypeptides encoded by a CD91 fragment nucleic acid sequences described in Section 5.1, above. In addition, CD91 polypeptide fragments of the invention may include proteins that represent functionally equivalent (see Section 5.1 for a definition) gene products. Such an equivalent CD91 polypeptide fragments may contain deletions, including internal deletions, additions, including additions yielding fusion proteins, or substitutions of amino acid residues within and/or adjacent to the amino acid sequence encoded by the gene sequences encoding CD91 polypeptide fragments described, above, in Section 5.1, but that result in a “silent” change, in that the change produces a functionally equivalent product encoded by the fragment of the CD91 gene. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Alternatively, where alteration of function is desired, deletion or non-conservative alterations can be engineered to produce altered CD91 polypeptide fragments. Such alterations can, for example, alter one or more of the biological functions of the CD91 polypeptide fragments. Further, such alterations can be selected so as to generate CD91 polypeptide fragments that are better suited for expression, scale up, etc. in the host cells chosen. For example, cysteine residues can be deleted or substituted with another amino acid residue in order to eliminate disulfide bridges.

Peptides and/or proteins corresponding to one or more domains of a CD91 polypeptide fragment as well as fusion proteins in which CD91 polypeptide fragments or portions of a CD91 polypeptide fragments such as a truncated CD91 polypeptide fragments or a peptide or a domain, is fused to an unrelated protein are also within the scope of this invention. Related proteins can also be fused with, for example domains from CD91 polypeptides derived from different organisms or non-contiguous domains of CD91. Such proteins and peptides can be designed on the basis of the CD91 fragment nucleotide sequences disclosed in Section 5.1, above, and/or on the basis of the CD91 polypeptide fragment amino acid sequence disclosed herein. Fusion proteins include, but are not limited to, IgFc fusions which stabilize the CD91 polypeptide fragments and prolong half life in vivo; or fusions to any amino acid sequence that allows the fusion protein to be anchored to the cell membrane; or fusions of CD91 polypeptide fragment domains to an enzyme, fluorescent protein, luminescent protein, or a flag epitope protein or peptide which provides a marker function.

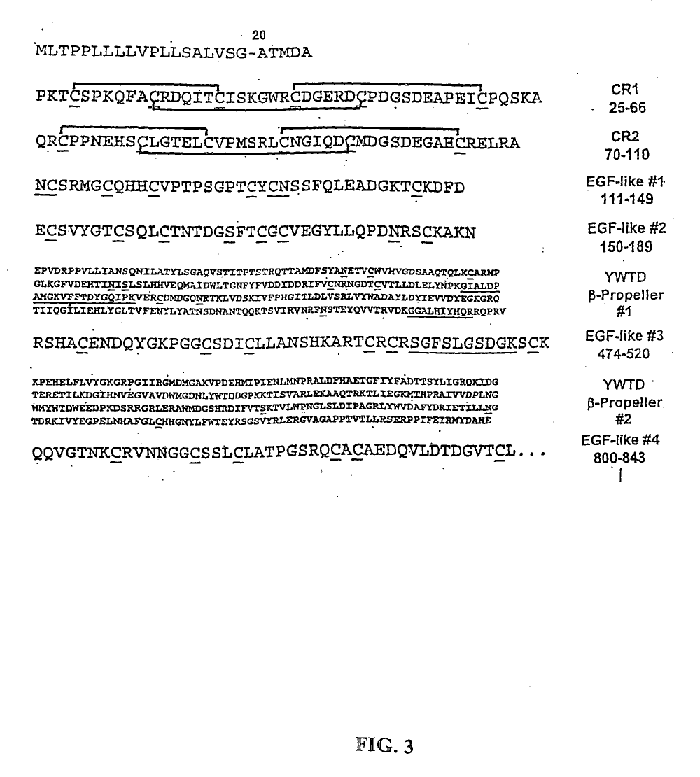

p95 polypeptides of the invention comprise residues 1-851 of SEQ ID NO:5. p95 polypeptide sequences can also include domains encoded by at least one exon of the cDNA sequence, or fragments thereof. Such domains include, but are not limited, for example, the HSP binding domain, the β-propeller #1 domain, the #1-propeller #2 domain, the CR1 domain, the CR2 domain, the EGF-like domain #1, the EGF-like domain #2, the EGF-like domain #3, and the EGF-like domain #4 (as shown in the mouse amino acid sequence of p80 in FIG. 3).

In certain embodiments of the invention, the CD9.1 polypeptide fragment of the invention comprises amino acid residues corresponding to Domain I and at least a portion of Domain II of a CD91 protein. In certain embodiments of the invention, the CD91 polypeptide fragment of the invention comprises at least amino acid residues 1-852 of the human CD91 protein. In other embodiments, the CD91 polypeptide fragment of the invention comprises amino acids from 1 through 853, 890, 894, 931, 935, 971, 975, 1011, 1014, 1051, 1061, 1097, 1103, 1140, 1143, 1182, 1184, 1221, 1226, 1261, 1268, 1531, 1539, 1578, 1582, 1846, 1849, 1886, 1932, 2151, 2154, 2194, 2198, 2473, 2481, or 2517 of the human CD91 protein.

In certain embodiments of the invention, the CD91 polypeptide fragment of the invention comprises amino acid residues corresponding to domain I, domain II, and at least a portion of domain III of a CD91 protein. In certain embodiments of the invention, the CD91 polypeptide fragment of the invention comprises at least amino acid residues 1-2518 of the human CD91 protein. In other embodiments, the CD91 polypeptide fragment of the invention comprises amino acids from 1 through 2523, 2561, 2565, 2600, 2604, 2639, 2651, 2688, 2695, 2730, 2733, 2769, 2773, 2812, 2817, 2853, 2857, 2897, 2903, 2939, 2943, 2980, 2985, 3021, 3028, 3284, 3293, or 3330 of the human CD91 protein.

In certain embodiments of the invention, the CD91 polypeptide fragment of the invention comprises amino acid residues corresponding to domain I, domain II, domain III and at least a portion of domain IV of a CD91 protein. In certain embodiments of the invention, the CD91 polypeptide fragment of the invention comprises at least amino acid residues 1-3331 of the human CD91 protein. In other embodiments, the CD91 polypeptide fragment of the invention comprises amino acids from 1 through 3333, 3369, 3373, 3408, 3412, 3448, 3452, 3489, 3493, 3531, 3535, 3570, 3574, 3609, 3612, 3647, 3653, 3690, 3694, 3731, 3740, 3776, 3784, 3822, 3827, 3843, 3860, 3867, 4075, 1425, 4142, 4150, 4182, 4199, 4231, 4235, 4267, 4271, 4278, 4303, 4307, 4339, 4343, 4374, 4376, 4408, or 4420 of the human CD91 protein.

In preferred embodiments, the CD91 polypeptide fragment comprises amino acid resides 1-986 (p110) or amino acid residues 1-2517 (p282) of SEQ ID NO:5. In other preferred embodiments, the CD91 polypeptide fragment comprises amino acid resides that comprise domain I and II of the receptor, wherein the receptor is a CD91 (alpha (2) macroglobulin receptor). In certain preferred embodiments, the receptor is from a mammal. In yet other preferred embodiments, the CD91 polypeptide fragment comprises amino acid resides that comprise domain I and contiguous complement repeats of the CD91, wherein the receptor is from a non-human origin. Such complement repeats or other regions may extend the sequence beyond domain I through complement repeats that correspond to CR3 of human CD91 (amino acid residues 854-890), CR4 of human CD91 (amino acid residues 895-931), CR5 of human CD91 (amino acid residues 936-971), or CR6 of human CD91 (amino acid residues 976-1011). In other embodiments, the CD91 polypeptide fragment comprises an amino acid sequence corresponding to domain I of a CD91 and the following 5, 10, 15, 20, 25, 30, 35, 40, 45, 50, 55, 60, 65, 70, 75, 80, 85, 90, 95, 100, 105, 110, 115, 120, 125, 130, 135, 140, 145, or 150 contiguous amino acids of domain II of a CD91.

In certain embodiments of the invention, the CD91 polypeptide fragment of the invention comprises amino acid residues 1-851 of the human CD91 protein and at least one of the defined regions selected from the group consisting of amino acid residues 854-890, 895-931, 936-971, 976-1011, 1015-1051, 1062-1097, 1104-1140, 1145-1182, 1185-1221, 1227-1261, 1269-1531, 1540-1578, 1583-1846, 1850-1886, 1934-2151, 2159-2194, 2199-2473, 2482-2517, 2524-2561, 2566-2600, 2605-2639, 2652-2688, 2696-2730, 2734-2769, 2774-2812, 2818-2853, 2858-2897, 2904-2939, 2944-2980, 2986-3021, 3029-3284, 3294-3330, 3334-3369, 3374-3408, 3413-3448, 3453-3489, 3494-3531, 3536-3570, 3575-3609, 3613-3647, 3654-3690, 3695-3731, 3741-3776, 3785-3822, 3828-3860, 3868-4142, 4151-4182, 4200-4231, 4236-4267, 4272-4303, 4308-4339, 4344-4374, 4377-4408, 4421-4444, and 4445-4544 of the human CD91 protein.

In other embodiments, CD91 polypeptide fragments refer to CD91 polypeptide sequences that comprise amino acid residue 1 through at least amino acid residue 851, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995, 2000, 2005, 2010, 2015, 2020, 2025, 2030, 2035, 2040, 2045, 2050, 2055, 2060, 2065, 2070, 2075, 2080, 2085, 2090, 2095, 2100, 2105, 2110, 2115, 2120, 2125, 2130, 2135, 2140, 2145, 2150, 2155, 2160, 2165, 2170, 2175, 2180, 2185, 2190, 2195, 2200, 2205, 2210, 2215, 2220, 2225, 2230, 2235, 2240, 2245, 2250, 2255, 2260, 2265, 2270, 2275, 2280, 2285, 2290, 2295, 2300, 2305, 2310, 2315, 2320, 2325, 2330, 2335, 2340, 2345, 2350, 2355, 2360, 2365, 2370, 2375, 2380, 2385, 2390, 2395, 2400, 2405, 2410, 2415, 2420, 2425, 2430, 2435, 2440, 2445, 2450, 2455, 2460, 2465, 2470, 2475, 2480, 2485, 2490, 2495, 2500, 2505, 2510, 2515, 2520, 2525, 2530, 2535, 2540, 2545, 2550, 2555, 2560, 2565, 2570, 2575, 2580, 2585, 2590, 2595, 2600, 2605, 2610, 2615, 2620, 2625, 2630, 2635, 2640, 2645, 2650, 2655, 2660, 2665, 2670, 2675, 2680, 2685, 2690, 2695, 2700, 2705, 2710, 2715, 2720, 2725, 2730, 2735, 2740, 2745, 2750, 2755, 2760, 2765, 2770, 2775, 2780, 2785, 2790, 2795, 2800, 2805, 2810, 2815, 2820, 2825, 2830, 2835, 2840, 2845, 2850, 2855, 2860, 2865, 2870, 2875, 2880, 2885, 2890, 2895, 2900, 2905, 2910, 2915, 2920, 2925, 2930, 2935, 2940, 2945, 2950, 2955, 2960, 2965, 2970, 2975, 2980, 2985, 2990, 2995, 3000, 3005, 3010, 3015, 3020, 3025, 3030, 3035, 3040, 3045, 3050, 3055, 3060, 3065, 3070, 3075, 3080, 3085, 3090, 3095, 3100, 3105, 3110, 3115, 3120, 3125, 3130, 3135, 3140, 3145, 3150, 3155, 3160, 3165, 3170, 3175, 3180, 3185, 3190, 3195, 3200, 3205, 3210, 3215, 3220, 3225, 3230, 3235, 3240, 3245, 3250, 3255, 3260, 3265, 3270, 3275, 3280, 3285, 3290, 3295, 3300, 3305, 3310, 3315, 3320, 3325, 3330, 3335, 3340, 3345, 3350, 3355, 3360, 3365, 3370, 3375, 3380, 3385, 3390, 3395, 3400, 3405, 3410, 3415, 3420, 3425, 3430, 3435, 3440, 3445, 3450, 3455, 3460, 3465, 3470, 3475, 3480, 3485, 3490, 3495, 3500, 3505, 3510, 3515, 3520, 3525, 3530, 3535, 3540, 3545, 3550, 3555, 3560, 3565, 3570, 3575, 3580, 3585, 3590, 3595, 3600, 3605, 3610, 3615, 3620, 3625, 3630, 3635, 3640, 3645, 3650, 3655, 3660, 3665, 3670, 3675, 3680, 3685, 3690, 3695, 3700, 3705, 3710, 3715, 3720, 3725, 3730, 3735, 3740, 3745, 3750, 3755, 3760, 3765, 3770, 3775, 3780, 3785, 3790, 3795, 3800, 3805, 3810, 3815, 3820, 3825, 3830, 3835, 3840, 3845, 3850, 3855, 3860, 3865, 3870, 3875, 3880, 3885, 3890, 3895, 3900, 3905, 3910, 3915, 3920, 3925, 3930, 3935, 3940, 3945, 3950, 3955, 3960, 3965, 3970, 3975, 3980, 3985, 3990, 3995, 4000, 4005, 4010, 4015, 4020, 4025, 4030, 4035, 4040, 4045, 4050, 4055, 4060, 4065, 4070, 4075, 4080, 4085, 4090, 4095, 4100, 4105, 4110, 4115, 4120, 4125, 4130, 4135, 4140, 4145, 4150, 4155, 4160, 4165, 4170, 4175, 4180, 4185, 4190, 4195, 4200, 4205, 4210, 4215, 4220, 4225, 4230, 4235, 4240, 4245, 4250, 4255, 4260, 4265, 4270, 4275, 4280, 4285, 4290, 4295, 4300, 4305, 4310, 4315, 4320, 4325, 4330, 4335, 4340, 4345, 4350, 4355, 4360, 4365, 4370, 4375, 4380, 4385, 4390, 4395, 4400, 4405, 4410, 4415, 4420, 4425, 4430, 4435, 4440, 4445, 4450, 4455, 4460, 4465, 4470, 4475, 4480, 4485, 4490, 4495, 4500, 4505, 4510, 4515, 4520, 4525, 4530, 4535, or 4540.

The CD91 polypeptide fragment of the invention can further comprise posttranslational modifications, including, but not limited to glycosylations, acetylations, myristylations, and phosphorylations. If the native CD91 polypeptide fragment does not have recognition motifs that allow such modifications, it would be routine for one skilled in the art to introduce into a gene nucleotide sequences that encode motifs such as enzyme recognition signals so as to produce a modified CD91 fragment gene product.

The CD91 polypeptide fragments and fusion proteins thereof, may be produced by recombinant DNA technology using techniques well known in the art. Thus, methods for preparing the CD91 fragment polypeptides, peptides, fusion peptide and fusion polypeptides of the invention by expressing nucleic acid containing CD91 gene sequences are described herein. Methods that are well known to those skilled in the art can be used to construct expression vectors containing CD91 gene-polypeptide fragment product coding sequences and appropriate transcriptional and translational control signals. These methods include, for example, in vitro recombinant DNA techniques, synthetic techniques, and in vivo genetic recombination. See, for example, the techniques described in Sambrook, et al, 1989, supra, and Ausubel, et al., 1989, supra. Alternatively, RNA capable of encoding CD91 fragment gene product sequences may be chemically synthesized using, for example, synthesizers. See, for example, the techniques described in “Oligonucleotide Synthesis”, 1984, Gait, ed., IRL Press, Oxford.

5.1.2.1 Variants, Derivatives, and Analogs of CD91 Polypeptide Fragments

CD91 polypeptide fragment derivatives can be made by altering CD91 polypeptide fragment coding sequences by substitutions, additions or deletions that provide for functionally equivalent molecules. Due to the degeneracy of nucleotide coding sequences, other DNA sequences which encode substantially the same amino acid sequence as a CD91 polypeptide fragment encoding nucleic acid may be used in the practice of the present invention. These include but are not limited to nucleotide sequences comprising all or CD91 polypeptide fragment-binding portions of a CD91 polypeptide fragment encoding nucleic acid which are altered by the substitution of different codons that encode a functionally equivalent amino acid residue within the sequence, thus producing a silent change. Likewise, the CD91 polypeptide fragment derivatives of the invention include, but are not limited to, those containing, as a primary amino acid sequence, all or a CD91 polypeptide fragment-binding portion of the amino acid sequence of a CD91 polypeptide fragment, including altered sequences in which functionally equivalent amino acid residues are substituted for residues within the sequence resulting in a silent change. In certain embodiments of the invention the CD91 polypeptide fragment derivative comprises about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 amino acid substitutions.

For example, one or more amino acid residues within the C-terminal of a peptide or a N-terminus protecting group can be substituted. An amino acid residue can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Additionally, D-amino acids may be used. The term “D-amino acid” as used herein, refers to an amino acid having a D-configuration. A D-amino acid may be a naturally occurring amino acid or an unnatural amino acid. Thus, polypeptides of the invention may comprise D-amino acids, a combination of D- and L-amino acids, and various “designer” amino acids (e.g., β-methyl amino acids, Cα-methyl amino acids, and Na-methyl amino acids, etc.) to convey special properties. Synthetic amino acids include ornithine for lysine, fluorophenylalanine for phenylalanine, and norleucine for leucine or isoleucine. Substitutions can also be made using phosphorous analogs of amino acids, such as α-amino phosphonic acids and α-amino phosphinic acids, or amino acids having non-peptide linkages, nucleic acids, nucleic acid analogs such as phosphorothioates or peptide nucleic acids (“PNAs”), hormones, antigens, synthetic or naturally occurring drugs, opiates, dopamine, serotonin, catecholamines, thrombin, acetylcholine, prostaglandins, organic molecules, pheromones, adenosine, sucrose, glucose, lactose and galactose.

The CD91 polypeptide fragment derivatives and analogs of the invention can be produced by various methods known in the art. The manipulations which result in their production can occur at the nucleic acid or protein level. For example, the cloned CD91 fragment nucleic acid sequence can be modified by any of numerous strategies known in the art (Maniatis, T., 1990, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the nucleic acid encoding a derivative or analog of CD91 polypeptide fragment, care should be taken to ensure that the modified gene remains within the same translational reading frame as a CD91 fragment, uninterrupted by translational stop signals, in the region of the nucleic acid where the desired CD91 polypeptide fragment activity is encoded.

Manipulations of the CD91 polypeptide fragment sequence may also be made at the protein level. Included within the scope of the invention are CD91 polypeptide fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

5.1.3 Recombinant Expression

In various embodiments of the invention, sequences encoding a CD91 polypeptide fragment, an HSP, or a CD91 ligand are inserted into an expression vector for propagation and expression in recombinant cells. Thus, in one embodiment, a CD91 polypeptide fragment, HSP, or a CD91 ligand coding region is linked to a non-native promoter for expression in recombinant cells.

The amino acid sequence of soluble recombinant human CD91 corresponds to SEQ ID NO:3. Based on this invention and the knowledge that HSPs play a role in immune responses, compositions comprising agonists and antagonists of CD91 polypeptide fragment and HSPs interactions can be used to modulate the immune response. Thus, recombinant CD91 polypeptide fragments, complexes of CD91 polypeptide fragments and an HSP or HSP-antigenic peptide complexes, and recombinant cells expressing a CD91 polypeptide fragments or complexes comprising a CD91 polypeptide fragments and peptides can be used in methods for immunotherapy and diagnostic methods described herein.

In various embodiments of the invention, sequences encoding a CD91 polypeptide fragment, and/or a heat shock protein, or fragments thereof, are inserted into an expression vector for propagation and expression in recombinant cells. An expression construct, as used herein, refers to a nucleotide sequence encoding a particular gene product, such as a CD91 polypeptide fragment or HSP, operably associated with one or more regulatory regions which allows expression of the encoded gene product in an appropriate host cell. “Operably-associated” refers to an association in which the regulatory regions and the nucleotide sequence encoding the gene product to be expressed are joined and positioned in such a way as to permit transcription, and ultimately, translation. In any of the embodiments disclosed herein, cells recombinantly expressing a CD91 polypeptide fragment may also be engineered to suppress or inhibit expression of endogenous CD91.

Alternatively, cell lines lacking CD91 or having mutant or non-functional CD91 can be employed. A nucleic acid encoding a CD91 polypeptide fragment may be used to alter the activity or expression of CD91. An antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate (S-ODNs), a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-atanomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15, 6625-6641). The oligonucleotide is a 2′-O-methylribonucleotide (Inoue et al., 1987, Nucl. Acids Res. 15, 6131-6148), or a chimeric RNA-DNA analogue (Inoue et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16, 3209), methylphosphonate oligonucleotides can be prepared by use of controlled pore glass polymer supports (Sarin et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred.

The DNA may be obtained from known sequences derived from sequence databases by standard procedures known in the art by DNA amplification or molecular cloning directly from a tissue, cell culture, or cloned DNA (e.g., a DNA “library”). Any eukaryotic cell may serve as the nucleic acid source for obtaining the coding region of an HSP encoding gene. Nucleic acid sequences encoding HSPs can be isolated from vertebrate, mammalian, as well as primate sources, including humans. Clones derived from genomic DNA may contain regulatory and intron DNA regions in addition to coding regions; clones derived from cDNA will contain only exon sequences. Whatever the source, the HSP gene should be cloned into a suitable vector for propagation of the gene.

Vectors based on E. coli are the most popular and versatile systems for high level expression of foreign proteins (Makrides, 1996, Microbiol Rev, 60:512-538). Non-limiting examples of regulatory regions that can be used for expression in E. coli may include but not limited to lac, trp, lpp, phoA, recA, tac, λP_{L}, and phage T3 and T7 promoters (Makrides, 1996, Microbiol Rev, 60:512-538). Non-limiting examples of prokaryotic expression vectors may include the λgt vector series such as λgt11 (Huynh et al., 1984 in “DNA Cloning Techniques”, Vol. 1: A Practical Approach (D. Glover, ed.), pp. 49-78, IRL Press, Oxford), and the pET vector series (Studier et al., 1990, Methods Enzymol., 185:60-89). However, a potential drawback of a prokaryotic host-vector system is the inability to perform many of the post-translational processing events of mammalian cells. Thus, an eukaryotic host-vector system is preferred, a mammalian host-vector system is more preferred, and a human host-vector system is the most preferred.

The regulatory regions necessary for transcription of a CD91 fragment sequence, for example, can be provided by the expression vector. A translation initiation codon (ATG) may also be provided to express a nucleotide sequence encoding a CD91 polypeptide fragment that lacks an initiation codon. In a compatible host-construct system, cellular proteins required for transcription, such as RNA polymerase and transcription factors, will bind to the regulatory regions on the expression construct to effect transcription of a CD91 fragment sequence in the host organism. The precise nature of the regulatory regions needed for gene expression may vary from host cell to host cell. Generally, a promoter is required which is capable of binding RNA polymerase to initiate the transcription of an operably-associated nucleic acid sequence. Such regulatory regions may include those 5′-non-coding sequences involved with initiation of transcription and translation, such as the TATA box, the cap site, a CAAT box, and the like. The non-coding region 3′ to the coding sequence may contain transcriptional termination regulatory sequences, such as terminators and polyadenylation sites.

Both constitutive and inducible regulatory regions may be used for expression of a CD91 polypeptide fragment, HSP, or a CD91 ligand. It may be desirable to use inducible promoters when the conditions optimal for growth of the recombinant cells and the conditions for high level expression of the gene product are different. Examples of useful regulatory regions are provided in the next section below.

For expression of a CD91 polypeptide fragment, HSP, or a CD91 ligand gene product in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the α-interferon gene, and the HSP70 gene (Williams et al., 1989, Cancer Res. 49:2735-42; Taylor et al., 1990, Mol. Cell Biol., 10:165-75). It may be advantageous to use heat shock promoters or stress promoters to drive expression of a CD91 fragment in recombinant host cells.

The following animal regulatory regions, which exhibit tissue specificity and have been utilized in transgenic animals, can also be used in tumor cells of a particular tissue type: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

The efficiency of expression of a CD91 fragment in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. in Biotechnol. 1:36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding a CD91 polypeptide fragment. For long term, high yield production of a CD91 polypeptide fragment, stable expression in mammalian cells is preferred. A number of selection systems may be used for mammalian cells, including, but not limited, to the Herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

In order to insert the DNA sequence encoding a CD91 polypeptide fragment, HSP, or a CD91 ligand into the cloning site of a vector, DNA sequences with regulatory functions, such as promoters, must be attached to DNA sequences encoding a CD91 polypeptide fragment, HSP, or a CD91 ligand, respectively. To do this, linkers or adapters providing the appropriate compatible restriction sites may be ligated to the ends of cDNA or synthetic DNA encoding a CD91 polypeptide fragment, by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

In one embodiment, an expression construct comprising a CD91 polypeptide fragment sequence operably associated with regulatory regions can be directly introduced into appropriate host cells for expression and production of a CD91 polypeptide fragment without further cloning (see, for example, U.S. Pat. No. 5,580,859). The expression constructs may also contain DNA sequences that facilitate integration of a CD91 fragment sequence into the genome of the host cell, e.g., via homologous recombination. In this instance, it is not necessary to employ an expression vector comprising a replication origin suitable for appropriate host cells in order to propagate and express a CD91 polypeptide fragment in the host cells.

Expression constructs containing cloned nucleotide sequence encoding a CD91 polypeptide fragment, an HSP, or other CD91 ligand, can be introduced into the host cell by a variety of techniques known in the art, including but not limited to, for prokaryotic cells, bacterial transformation (Hanahan, 1985, in DNA Cloning, A Practical Approach, 1:109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479-488).

For long term, high yield production of a properly processed CD91 polypeptide fragment, HSP, or a CD91 ligand, stable expression in mammalian cells is preferred. Cell lines that stably express a CD91 polypeptide fragment, HSP, or a CD91 ligand or a CD91 polypeptide fragment-peptide complex may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the desired gene product is expressed continuously.

The recombinant cells may be cultured under standard conditions of temperature, incubation time, optical density, and media composition. Alternatively, recombinant antigenic cells may be cultured under conditions emulating the nutritional and physiological requirements of the cancer cell or infected cell. However, conditions for growth of recombinant cells may be different from those for expression of a CD91 polypeptide fragment, HSPs, or a CD91 ligand, or antigenic peptide.

5.1.4 Peptide Synthesis

An alternative to producing peptides and polypeptides comprising HSP, a CD91 polypeptide fragment or CD91 ligand sequences by recombinant techniques is peptide synthesis. For example, a peptide corresponding to a portion of an HSP or a CD91 polypeptide fragment comprising the receptor-binding domain, which can be used as an antagonist in the therapeutic methods described herein, can be synthesized by use of a peptide synthesizer. Peptides corresponding to CD91 polypeptide fragment sequences useful for therapeutic methods described herein can also be produced synthetically. Conventional peptide synthesis may be used or other synthetic protocols well known in the art.

For example, peptides having the amino acid sequence of a CD91 polypeptide fragment, an HSP, or a CD91 ligand, or an analog, mutein, fragment, or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support, i.e., polystyrene beads. The peptides are synthesized by lnking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CD91 polypeptide fragment, HSP, or CD91 ligand peptide is accomplished using conventional procedures, such as preparative HPLC using gel permeation, partition and/or ion exchange chromatography. The choice of appropriate matrices and buffers are well known in the art and so are not described in detail herein.

In addition, analogs and derivatives of a CD91 polypeptide fragment, HSP, or a CD91 ligand protein can be chemically synthesized. Furthermore, if desired, nonclassical amino acids or chemical amino acid analogs can be introduced as a substitution or addition into a CD91 polypeptide fragment, HSP, or a CD91 ligand sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

5.1.5 Antibodies Specific for CD91 Polypeptide Fragments and CD91 Polypeptide Fragment-HSP Complexes

Described herein are methods for the production of antibodies capable of specifically recognizing CD91 polypeptide fragment epitopes, HSP-CD91 polypeptide fragment complex epitopes or epitopes of conserved variants or peptide fragments of the receptor or receptor complexes. Such antibodies are useful for recognizing the HSP-binding domain of CD91 for use in therapeutic and diagnostic methods of the invention.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a CD91 polypeptide fragment or HSP-CD91 polypeptide fragment complex in an biological sample. Such antibodies may also be utilized in conjunction with, for example, compound screening schemes, as described below, in Section 5.2, for the evaluation of the effect of test compounds on the interaction between HSPs and the CD91 fragment protein. The interaction between HSPs and the CD91 fragment protein can be an immunospecific binding of the two.

Anti-CD91 fragment protein complex antibodies may additionally be used as a method for the inhibition of abnormal CD91 product activity. Thus, such antibodies may, be utilized as part of treatment methods for HSP-CD91 related disorders, e.g., autoimmune disorders.

For the production of antibodies against CD91 fragment protein or peptide complexes thereof, various host animals may be immunized by injection with a CD91 fragment protein or HSP-CD91 fragment protein complex, or a portion thereof. An antigenic portion of CD91 fragment protein or HSP-CD91 fragment protein complex can be readily predicted by algorithms known in the art. In certain embodiments, anti-CD91 polypeptide fragment antibodies may be generated to certain CD91 polypeptide fragments such as, but not limited to, p95, p110, p282, p373, or p494. Such antibodies may be utilized as part of the treatment methods of the invention for HSP-CD91 related disorders.

Host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a CD91 polypeptide fragment protein or HSP-CD91 polypeptide fragment protein complex, or an antigenic functional derivative thereof. For the production of polyclonal antibodies, host animals such as those described above, may be immunized by injection with CD91 polypeptide fragment protein or HSP-CD91 polypeptide fragment protein complex, or portion thereof, supplemented with adjuvants as also described above.

Monoclonal antibodies, which are homogeneous populations of antibodies to a particular antigen, may be obtained by any technique that provides the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81: 6851-6855; Neuberger, et al., 1984, Nature 312: 604-608; Takeda, et al., 1985, Nature, 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397, which are incorporated herein by reference in their entirety).

In an additional embodiment of the invention, monoclonal antibodies can be produced in germ-free animals (see PCT International Publication No. WO 89/12690, published Dec. 12, 1989). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96).

Techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for an CD91 polypeptide fragment protein-HSP complex together with genes from a human antibody molecule of appropriate biological activity can also be used; such antibodies are within the scope of this invention.

Humanized antibodies are also provided (see U.S. Pat. No. 5,225,539 by Winter). An immunoglobuin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and a framework region from a human immunoglobulin molecule. Such CDRS-grafted antibodies have been successfully constructed against various antigens, for example, antibodies against IL-2 receptor as described in Queen et al., 1989, Proc. Natl. Acad. Sci. USA 86:10029; antibodies against the cell surface receptor CAMPATH as described in Riechmann et al., 1988, Nature 332:323; antibodies against hepatitis B in Co et al., 1991, Proc. Natl. Acad. Sci. USA 88:2869; as well as against viral antigens of the respiratory syncytial virus in Tempest et al., 1991, Bio-Technology 9:267. Humanized antibodies are most preferred for therapeutic use in humans.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be adapted to produce single chain antibodies against CD91 polypeptide fragment protein or HSP-CD91 polypeptide fragment protein complexes, or portions thereof. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse et al., 1989, Science, 246: 1275-1281) to allow rapid and easy identification of monoclonal Fab fragments with the desired specificity.

Antibodies to the CD91 polypeptide fragment can, in turn, be utilized to generate anti-idiotype antibodies that “mimic” the CD91 polypeptide fragment, using techniques well known to those skilled in the art (see, e.g., Greenspan & Bona, 1993, FASEB J 7(5):437-444; and Nissinoff, 1991, J. Immunol. 147(8):2429-2438). For example antibodies which bind to the CD91 polypeptide fragment and competitively inhibit the binding of HSPs to the CD91 polypeptide fragment can be used to generate anti-idiotypes that “mimic” the ECD and, therefore, bind and neutralize HSPs. Such neutralizing anti-idiotypes or Fab fragments of such anti-idiotypes can be used in therapeutic regimens to neutralize the native ligand and treat HSP-CD91-related disorders, such as immunological disorders, proliferative disorders, and infectious diseases.

Alternatively, antibodies to a CD91 polypeptide fragment that can act as agonists of a CD91 polypeptide fragment activity or CD91 activity can be generated. Such antibodies will bind to a CD91 polypeptide fragment and CD91 and can potentially activate the signal transducing activity of the CD91 the activation of a CD91 would be particularly useful for treating proliferative disorders, such as cancer, and infectious diseases. In addition, antibodies that act as antagonist of a CD91 polypeptide fragment activity, i.e., potential inhibitors of the activation of a CD91, would be particularly useful for treating autoimmune disorders. Methods for assaying for such agonists and antagonists are described in detail in Section 5.2, below.

5.2 Assays for the Identification of Compounds that Interact with the CD91 Polypeptide Fragment

Methods for identifying compounds that interact with the CD91 polypeptide fragment, or enhance or block the function of the CD91 polypeptide fragment, are included in the invention. The present invention provides in vitro and in vivo assay systems, described in the subsections below, which can be used to identify compounds or compositions that interact with the CD91 polypeptide fragment, or modulate the activity of the CD91 polypeptide fragment and its interaction with HSPs or HSP-peptide complexes.

The invention provides screening methodologies useful in the identification of small molecules, proteins and other compounds which interact with the CD91 polypeptide fragment, or modulate the interaction of HSPs with the CD91 polypeptide fragment. Such compounds may bind the CD91 polypeptide fragment genes or gene products with differing affinities, and may serve as regulators of receptor activity in vivo with useful therapeutic applications in modulating the immune response. For example, certain compounds that inhibit receptor function may be used in patients to downregulate destructive immune responses which are caused by cellular release of HSPs. In one embodiment, the compound is an agonist which enhances the interaction of the heat shock protein and a CD91 polypeptide fragment. In another embodiment of this method the compound in an antagonist that interferes with the interaction between the heat shock protein and a CD91 polypeptide fragment.

Methods to screen potential agents for their ability to interact with the CD91 polypeptide fragment, or modulate CD91 polypeptide fragment expression and activity can be designed based on the Applicant's discovery of the receptor and its role in HSP or HSP-peptide complex binding and recognition. CD91 polypeptide fragments, nucleic acids, and derivatives can be used in screening assays to detect molecules that specifically bind to CD91 or HSP proteins, derivatives, or nucleic acids, and thus have potential use to modulate the CD91 HSP interactions and thereby modulate an immune response. In a preferred embodiment, such assays are performed to screen for molecules with potential utility as anti-autoimmune disease, anti-cancer, anti-infective drugs, and immunomodulatory drugs (such as anti-viral drugs and antibiotic drugs), or lead compounds for drug development. For example, recombinant cells expressing CD91 fragment nucleic acids can be used to recombinantly produce CD91 polypeptide fragment protein for use in these assays, to screen for molecules that interfere with the binding of HSPs to the CD91 polypeptide fragment. Similar methods can be used to screen for molecules that bind to the CD91 polypeptide fragment derivatives or encoding nucleic acids. Methods that can be used to carry out the foregoing are commonly known in the art.

Compounds capable of specifically binding the CD91 polypeptide fragment protein can be useful for immunotherapy. In one embodiment, an assay is disclosed for identifying compounds that specifically bind the CD91 polypeptide fragment protein comprising: (a) contacting a CD91 polypeptide fragment protein with one or more test compounds under conditions conducive to binding; and (b) identifying one or more test compounds which specifically bind to the CD91 polypeptide fragment protein, such that a compound capable of specifically binding the CD91 polypeptide fragment protein is identified as a compound useful for immunotherapy.

Another method encompassed by the invention for identifying a compound useful for immunotherapy involves identifying a compound which modulates the binding of an CD91 polypeptide fragment ligand to the CD91 polypeptide fragment. The term “CD91 polypeptide fragment ligand” as used herein, refers to an molecule capable of binding to the CD91 polypeptide fragment. Such CD91 polypeptide fragment ligands include, but are not limited to, heat shock proteins and heat shock protein complexes.

The method comprises the steps of: (a) contacting a CD91 polypeptide fragment with a CD91 polypeptide fragment ligand, or analog, derivative or mimetic thereof, in the presence of one or more test compound; and (b) measuring the amount of CD91 polypeptide fragment ligand, or analog, derivative or mimetic thereof, bound to the CD91 polypeptide fragment, such that if the amount of bound CD91 polypeptide fragment ligand measured in (b) differs from the amount of bound CD91 polypeptide fragment measured in the absence of the test compound, then a compound can be useful for immunotherapy that modulates the binding of a CD91 polypeptide fragment ligand to the CD91 polypeptide fragment is identified.

In another embodiment, a method for identifying a compound useful for immunotherapy which modulates the interaction between the CD91 polypeptide fragment and an CD91 polypeptide fragment ligand is provided by the invention. This method comprises the steps of: (a) contacting an CD91 polypeptide fragment with one or more test compounds; and (b) measuring the level of CD91 polypeptide fragment activity or expression, such that if the level of activity or expression measured in (b) differs from the level of CD91 polypeptide fragment activity in the absence of one or more test compounds, then a compound that modulates the interaction between the CD91 polypeptide fragment and an CD91 polypeptide fragment ligand is identified.

In another embodiment, an assay for identifying a compound that modulates an HSP-CD91 polypeptide fragment-mediated process is disclosed. This assay comprises: (a) contacting a test compound with an HSP and a CD91 polypeptide fragment; and (b) measuring the level of CD91 polypeptide fragment activity or expression, such that if the level of activity or expression measured in (b) differs from the level of CD91 polypeptide fragment activity in the absence of the test compound, then a compound that modulates an HSP-CD91 polypeptide fragment-mediated process is identified. In another embodiment, in which the compound identified is an antagonist which interferes with the interaction of the HSP with the CD91 polypeptide fragment, the method further comprises the step of determining whether the level interferes with the interaction of the HSP and the CD91 polypeptide fragment.

In another embodiment, a cell-based method for identifying a compound that modulates an HSP-CD91 polypeptide fragment-mediated process is described. This method comprises the following steps: (a) contacting a test compound with a heat shock protein and an CD91 polypeptide fragment-expressing cell; and (b) measuring the level of CD91 polypeptide fragment activity or expression in the cell, such that if the level of activity or expression measured in (b) differs from the level of CD91 polypeptide fragment activity in the absence of the test compound, then a compound that modulates an HSP-CD91 polypeptide fragment protein-mediated process is identified.

In another embodiment, a receptor-ligand binding assay for identifying a compound that interacts with CD91 polypeptide fragment, or modulates the binding of an HSP to CD91 polypeptide fragment. One such method comprises: (a) contacting an HSP with an CD91 polypeptide fragment, or analog, derivative or mimetic thereof, in the presence of a test compound; and (b) measuring the amount of heat shock protein bound to the CD91 polypeptide fragment, or analog, derivative or mimetic thereof, such that if the amount of bound heat shock protein measured in (b) differs from the amount of bound heat shock protein measured in the absence of the test compound, then a compound that modulates the binding of an HSP to the CD91 polypeptide fragment is identified.

The assays of the present invention may be first optimized on a small scale (i.e., in test tubes), and then scaled up for high-throughput assays. In various embodiments, the in vitro screening assays of the present invention may be performed using purified components or cell lysates. In other embodiments, the screening assays may be carried out in intact cells in culture and in animal models. In accordance with the present invention, test compounds which are shown to modulate the activity of the CD91 polypeptide fragment as described herein in vitro, will further be assayed in vivo, including cultured cells and animal models to determine if the test compound has the similar effects in vivo, via interactions with endogenous CD91, and to determine the effects of the test compound on antigen presentation, cytokine release, intracellular Ca⁺⁺ release, T-cell cytotoxicity, tumor progression, the accumulation or degradation of positive and negative regulators, cellular proliferation, etc.

Proteins that are identified as interacting with a CD91 polypeptide fragment can be further identified and sequenced for use in the methods of the invention. Further identification can be by affinity chromatography, analyzed on SDS-PAGE, stained with coomasie blue or transferred onto PVDF membrane and stained with coomasie blue, all of it under keratin-free conditions. Protein bands can then be excised with a razor from the gel or membrane. Tryptic peptides from an 80 kDa faint coomassie band can be extracted by 50% acetonitrile, 5% formic acid, dried, and loaded onto a 75 m 10 cm, reverse-phase C18, microcapillary column (3 μl vol) and tryptic peptides can be separated by on-line microcapillary liquid chromatography-tendem mass spectrometry followed by database searching using the SEQUEST program as previously described. (Gatlin et al., 2000, Anal. Chem. 72:757-63; Link et al., 1999, Nat. Biotechnol. 17:676-82). The analysis can be carried out in a data-dependent auto-MS/MS fashion using a Finnigan LCQ iontrap Mass Spectrometer.

5.2.1 CD91 Polypeptide Fragment—Ligand Binding Assays

The screening assays, described herein, can be used to identify compounds and compositions, including peptides and organic, non-protein molecules that interact with a CD91 polypeptide fragment, or that modulate the interaction between HSPs and a CD91 polypeptide fragment. Recombinant, synthetic, and otherwise exogenous compounds may have binding capacity and, therefore, may be candidates for pharmaceutical agents. Alternatively, the proteins and compounds include endogenous cellular components which interact with the identified genes and proteins in vivo. Such endogenous components may provide new targets for pharmaceutical and therapeutic interventions.

Thus, in a preferred embodiment, both naturally occurring and/or synthetic compounds (e.g., libraries of small molecules or peptides), may be screened for interacting with a CD91 polypeptide fragment and/or modulating CD91 polypeptide fragment activity. In another series of embodiments, cell lysates or tissue homogenates may be screened for proteins or other compounds which bind to one of the normal or mutant CD91 fragment nucleic acids and CD91 polypeptide fragment.

The screening assays described herein may be used to identify small molecules, peptides or proteins, or derivatives, analogs and fragments thereof, that interact with and/or modulate the interaction of HSPs with a CD91 polypeptide fragment. Such compounds may be used as agonists or antagonists of the uptake of CD91 ligands, such as HSPs and HSP complexes, by the cell surface receptor. For example, compounds that modulate a CD91 polypeptide fragment-ligand interaction include, but are not limited to, compounds that bind to a CD91 polypeptide fragment, thereby either inhibiting (antagonists) or enhancing (agonists) the binding of ligands, such as HSPs and HSP complexes, to the receptor, as well as compounds that bind to the ligand, such as for example, HSPs, thereby preventing or enhancing binding of ligand to the receptor. Compounds that affect CD91 fragment DNA activity (by affecting CD91 fragment DNA expression, including molecules, e.g., proteins or small organic molecules, that affect transcription or interfere with splicing events so that expression of CD91 fragment can be modulated) can also be identified in the screens of the invention.

The screening assays described herein are designed to detect compounds that modulate, i.e., interfere with or enhance, ligand-CD91 polypeptide fragment interactions, including HSP-CD91 polypeptide fragment interactions. As described in detail below, such assays are functional assays, such as binding assays, that can be adapted to a high-throughput screening methodologies.

Binding assays can be used to identify compounds that modulate the interaction between ligands, for example, HSPs, and a CD91 polypeptide fragment. In one aspect of the invention the screens may be designed to identify compounds that disrupt the interaction between a CD91 polypeptide fragment and a ligand, such as, for example, HSPs or peptides derived from an HSP, or another CD91 ligand. Such compounds will be useful as lead compounds for antagonists of HSP-CD91-related disorders and conditions, such as immune disorders, proliferative disorders, and infectious diseases.

Binding assays may be performed either as direct binding assays or as competition binding assays. In a direct binding assay, a test compound is tested for binding either to a CD91 polypeptide fragment or to a CD91 ligand, such as an HSP. Then, in a second step, the test compound is tested for its ability to modulate the ligand-CD91 polypeptide fragment interaction. Competition binding assays, on the other hand, assess the ability of a test compound to compete with a ligand, i.e., an HSP, for binding to CD91 polypeptide fragment.

In a direct binding assay, either the ligand and/or CD91 polypeptide fragment is contacted with a test compound under conditions that allow binding of the test compound to the ligand or the receptor. The binding may take place in solution or on a solid surface. Preferably, the test compound is previously labeled for detection. Any detectable compound may be used for labeling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, it involves washing with an appropriate buffer. Finally, the presence of a ligand-test compound (e.g., HSP-test compound) or a CD91 polypeptide fragment-test compound complex is detected.

In a competition binding assay, test compounds are assayed for their ability to disrupt or enhance the binding of the ligand (e.g., HSP) to a CD91 polypeptide fragment. Labeled ligand (e.g., HSP) may be mixed with a CD91 polypeptide fragment or derivative thereof, and placed under conditions in which the interaction between them would normally occur, with and without the addition of the test compound. The amount of labeled ligand (e.g., HSP) that binds a CD91 polypeptide fragment may be compared to the amount bound in the presence or absence of test compound.

In a preferred embodiment, to facilitate complex formation and detection, the binding assay is carried out with one or more components immoblilized on a solid surface. In various embodiments, the solid support could be, but is not restricted to, polycarbonate, polystyrene, polypropylene, polyethlene, glass, nitrocellulose, dextran, nylon, polyacrylamide and agarose. The support configuration can include beads, membranes, microparticles, the interior surface of a reaction vessel such as a microtiter plate, test tube or other reaction vessel. The immobilization of a CD91 polypeptide fragment, or other component, can be achieved through covalent or non-covalent attachments. In one embodiment, the attachment may be indirect, i.e., through an attached antibody. In another embodiment, a CD91 polypeptide fragment and negative controls are tagged with an epitope, such as glutathione S-transferase (GST) so that the attachment to the solid surface can be mediated by a commercially available antibody such as anti-GST (Santa Cruz Biotechnology).

For example, such an affinity binding assay may be performed using a CD91 polypeptide fragment which is immobilized to a solid support. Typically, the non-mobilized component of the binding reaction, in this case either ligand (e.g., HSP) or the test compound, is labeled to enable detection. A variety of labeling methods are available and may be used, such as luminescent, chromophore, fluorescent, or radioactive isotope or group containing same, and nonisotopic labels, such as enzymes or dyes. In a preferred embodiment, the test compound is labeled with a fluorophore such as fluorescein isothiocyanate (FITC, available from Sigma Chemicals, St. Louis).

The labeled test compounds, or ligand (e.g., HSP) plus test compounds, are then allowed to contact with the solid support, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and so on.

Finally, the label remaining on the solid surface may be detected by any detection method known in the art. For example, if the test compound is labeled with a fluorophore, a fluorimeter may be used to detect complexes.

Preferably, the CD91 polypeptide fragment is added to binding assays in the form of intact cells that express the CD91 polypeptide fragment, or isolated membranes containing the CD91 polypeptide fragment. Thus, direct binding to the CD91 polypeptide fragment or the ability of a test compound to modulate a ligand-CD91 polypeptide fragment complex (e.g., HSP-CD91 polypeptide fragment complex) may be assayed in intact cells in culture or in animal models in the presence and absence of the test compound. A labeled ligand (e.g., HSP) may be mixed with cells that express the CD91 polypeptide fragment, or to crude extracts obtained from such cells, and the test compound may be added. Isolated membranes may be used to identify compounds that interact with the CD91 polypeptide fragment. For example, in a typical experiment using isolated membranes, cells may be genetically engineered to express the CD91 polypeptide fragment. Membranes can be harvested by standard techniques and used in an in vitro binding assay. Labeled ligand (e.g., ¹²⁵I-labeled HSP) is bound to the membranes and assayed for specific activity; specific binding is determined by comparison with binding assays performed in the presence of excess unlabeled (cold) ligand.

Alternatively, soluble CD91 polypeptide fragment may be recombinantly expressed and utilized in non-cell based assays to identify compounds that bind to the CD91 polypeptide fragment. The recombinantly expressed CD91 polypeptide fragment polypeptides or fusion proteins containing the extracellular domain (ECD) of the CD91 polypeptide fragment, or derivatives thereof, can be used in the non-cell based screening assays. In non-cell based assays the recombinantly expressed the CD91 polypeptide fragment is attached to a solid substrate such as a test tube, microtiter well or a column, by means well known to those in the art (see Ausubel et al., supra). The test compounds are then assayed for their ability to bind to the CD91 polypeptide fragment.

Alternatively, the binding reaction may be carried out in solution. In this assay, the labeled component is allowed to interact with its binding partner(s) in solution. If the size differences between the labeled component and its binding partner(s) permit such a separation, the separation can be achieved by passing the products of the binding reaction through an ultrafilter whose pores allow passage of unbound labeled component but not of its binding partner(s) or of labeled component bound to its partner(s). Separation can also be achieved using any reagent capable of capturing a binding partner of the labeled component from solution, such as an antibody against the binding partner, a ligand-binding protein which can interact with a ligand previously attached to the binding partner, and so on.

In a one embodiment, for example, a phage library can be screened by passing phage from a continuous phage display library through a column containing purified CD91 fragment, or derivative, analog, fragment, or domain, thereof, linked to a solid phase, such as plastic beads. By altering the stringency of the washing buffer, it is possible to enrich for phage that express peptides with high affinity for the CD91 polypeptide fragment. Phage isolated from the column can be cloned and the affinities of the short peptides can be measured directly. Sequences for more than one oligonucleotide can be combined to test for even higher affinity binding to the CD91 polypeptide fragment. Knowing which amino acid sequences confer the strongest binding to the CD91 polypeptide fragment, computer models can be used to identify the molecular contacts between the CD91 polypeptide fragment and the test compound. This will allow the design of non-protein compounds which mimic those contacts. Such a compound may have the same activity of the peptide and can be used therapeutically, having the advantage of being efficient and less costly to produce.

In another specific embodiment of this aspect of the invention, the solid support is membranes containing the CD91 polypeptide fragment attached to a microtiter dish. Test compounds, for example, cells that express library members are cultivated under conditions that allow expression of the library members in the microtiter dish. Library members that bind to the protein (or nucleic acid or derivative) are harvested. Such methods, are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment of the present invention, interactions between the CD91 polypeptide fragment or ligand (e.g., HSP) and a test compound may be assayed in vitro. Known or unknown molecules are assayed for specific binding to the CD91 fragment nucleic acids, proteins, or derivatives under conditions conducive to binding, and then molecules that specifically bind to the CD91 polypeptide fragment are identified. The two components can be measured in a variety of ways. One approach is to label one of the components with an easily detectable label, place it together with a test component(s) under conditions that allow binding to occur, perform a separation step which separates bound labeled component from unbound labeled component, and then measure the amount of bound component. In one embodiment, the CD91 polypeptide fragment can be labeled and added to a test agent, using conditions that allow binding to occur. Binding of the test agent can be determined using polyacrylamide gel analysis to compare complexes formed in the presence and absence of the test agent.

In other embodiments, antigen-specific response assays may be used to detect the effect of a candidate compound on presentation of antigenic molecule by an CD91 ligand, for example an HSP or HSP complex. Such assays may be in vitro or in vivo, and may encompass, for example, but not limited to, antigen representation assays, or tumor challenge or rejection assays to detect modulation of CD91 or HSP activity by the compounds identified by the methods of the invention. For example, an antigen presentation assay may be performed to determine the activity of a CD91 polypeptide fragment or the effect of a compound in vivo on the uptake of complexes capable of interacting with a CD91 polypeptide fragment, e.g., HSP-antigenic molecule complexes, by cells expressing the a CD91 polypeptide fragment. Such re-presentation assays are known in the art, and have been described previously (Suto and Srivastava, 1995, Science 269:1585-1588). For example, in one embodiment, antigen presenting cells, such as a macrophage cell line (e.g., RAW264.7), are mixed with antigen-specific T cells in media, using approximately 10,000 cells of each type at approximately a 1:1 ratio. Complexes of HSP (10 μg/ml) and a peptide antigen, as well as test compound, is added to the cells and the culture is incubated for approximately 20 hours. Stimulation of T cells may then be measured in the presence and absence of test compound.

In another embodiment, antigen-specific T cell stimulation may be assayed. In one embodiment an IFN-γ release assay may be used. After washing, cells are fixed, permeabilized, and reacted with dye-labeled antibodies reactive with human IFN-γ (PE-anti-IFN-γ). Samples are analyzed by flow cytometry using standard techniques. Alternatively, a filter immunoassay, ELISA (enzyme linked immunosorbent assay), or enzyme-linked immunospot assay (ELISPOT) assay, may be used to detect specific cytokines produced by an activated T cell. In one embodiment, for example, a nitrocellulose-backed microtiter plate is coated with a purified cytokine-specific primary antibody, i.e., anti-IFN-γ, and the plate is blocked to avoid background due to nonspecific binding of other proteins. A sample of APC cells stimulated with antigen is diluted onto the wells of the microtiter plate. A labeled, e.g., biotin-labeled, secondary anti-cytokine antibody is added. The antibody cytokine complex can then be detected, i.e., by enzyme-conjugated streptavidin-cytokine-secreting cells will appear as “spots” by visual, microscopic, or electronic detection methods. In another embodiment, “tetramer staining” assay (Altman et al., 1996, Science 274: 94-96) may be used to identify antigen-specific T-cells. For example, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of stimulated T cells. Biotin is then used to stain T cells which recognize and bind to the MHC-antigen complex.

5.2.2 Compounds that can be Screened in Accordance with the Invention

The screening assays described herein may be used to identify small molecules, peptides or proteins, or derivatives, analogs and fragments thereof, that interact with, or modulate the interaction of a ligand (e.g., HSP) with a CD91 polypeptide fragment. The compounds which may be screened in accordance with the invention include, but are not limited to small molecules, peptides, antibodies and fragments thereof, and other organic compounds (e.g., peptidomimetics) that bind to a CD91 polypeptide fragment and either inhibit the activity triggered by the natural ligand (i.e., antagonists) or mimic the activity triggered by the natural ligand (i.e., agonists), as well as small molecules, peptides, antibodies or fragments thereof, and other organic compounds. In another embodiment, such compounds include ligand sequences, such as HSP sequences, which can bind to the active site of a CD91 polypeptide fragment, and block its activity.

Compounds that may be used for screening include, but are not limited to, peptides such as, for example, soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab)₂ and FAb expression library fragments, and epitope-binding fragments thereof), and small organic or inorganic molecules.

In one embodiment of the present invention, peptide libraries may be used as a source of test compounds that can be used to screen for modulators of CD91 polypeptide fragment interactions, such as HSP-CD91 polypeptide fragment interactions. Diversity libraries, such as random or combinatorial peptide or nonpeptide libraries can be screened for molecules that specifically bind to the CD91 polypeptide fragment. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al, 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

In another embodiment of the present invention, the screening may be performed by adding the labeled ligand (e.g., HSP) to in vitro translation systems such as a rabbit reticulocyte lysate (RRL) system and then proceeding with in vitro priming reaction. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

Compounds that can be tested and identified methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, Germany), Takasago (Rockleigh, N.J.), SST Corporation (Clifton, N.J.), Ferro (Zachary, La. 70791), Riedel-deHaen Aktiengesellschaft (Seelze, Germany), PPG Industries Inc., Fine Chemicals (Pittsburgh, Pa. 15272). Further any kind of natural products may be screened using the methods of the invention, including microbial, fungal, plant or animal extracts.

Furthermore, diversity libraries of test compounds, including small molecule test compounds, may be utilized. Typically small molecules range in molecular weight from about 400 to 10,000 Daltons. For example, libraries may be commercially obtained from Specs and BioSpecs B.V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilize, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145). Combinatorial libraries of test compounds, including small molecule test compounds, can be utilized, and may, for example, be generated as disclosed in Eichler & Houghten, 1995, Mol. Med. Today 1:174-180; Dolle, 1997, Mol. Divers. 2:223-236; and Lam, 1997, Anticancer Drug Des. 12:145-167.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al, 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

5.3 Therapeutic Uses

The invention further encompasses methods for modulating the immune response. CD91 recognizes and transports antigenic peptide complexes (e.g., HSP-antigenic peptide complexes) for the purpose of presenting such antigenic molecules to cells of the immune system and eliciting an immune response. Thus, the CD91 polypeptide fragment compositions and methods of the invention may be used for therapeutic treatment of HSP-CD91-related disorders and conditions, such as autoimmune diseases, cancer and infectious diseases. In particular, as described in detail herein below, recombinant cells comprising CD91 polypeptide fragment, antibodies, and other compounds that interact with CD91, or modulate the interaction between CD91 and its ligands, e.g., HSP, as well as other compounds that modulate HSP-CD91-mediated processes may be used to elicit, or block, an immune response to treat such HSP-CD91-related disorders and conditions. Recombinant cells comprising CD91 polypeptide fragment, antibodies, and other compounds that interact with CD91, or modulate the interaction between CD91 and its ligands, e.g., HSP, as well as other compounds that modulate HSP-CD91-mediated processes may also be used to effect binding of other CD91 ligands and thereby impact lipid metabolism.

Compounds, such as those identified by screening methods provided herein, that modulate a CD91 ligand-CD91 polypeptide fragment interaction can be useful as therapeutics. Such compounds, include, but are not limited to, agonists, antagonists, such as antibodies, antisense RNAs and ribozymes. Compounds which interfere with ligand (e.g., HSP)-CD91 polypeptide fragment interaction can be used to modulate, i.e., increase or decrease an immune response, and can be used to treat autoimmune responses and conditions. Other antibodies, agonists, antagonists, antisense RNAs and ribozymes may upregulate ligand (e.g., HSP)-CD91 polypeptide fragment interaction, activity, or expression, and would inhibit or enhance the uptake of antigen complexes (e.g., HSP-antigen complexes) by CD91, and therefore be useful in stimulating the host's immune system prior to, or concurrent with, the administration of a vaccine. Described below are methods and compositions for the use of such compounds in the treatment of HSP-CD91-related disorders, such as immune disorders, proliferative disorders, and infectious diseases.

In one embodiment an antagonist of CD91 polypeptide fragment-ligand interaction is used to block the immune response. Such antagonists include compounds that interfere with binding of a ligand (e.g., an HSP) to a CD91 polypeptide fragment portion of a CD91 by competing for binding to CD91, the ligand, or a ligand-peptide complex.

In one embodiment, the antagonist is an antibody specific for the CD91 polypeptide fragment, or a fragment thereof which contains the HSP ligand binding site. In another embodiment the antagonist is an antibody specific for an HSP, which interferes with binding of the HSP to the receptor.

In another embodiment, the antagonist is an peptide which comprises at least contiguous 10 amino acids of an HSP sequence. Such a peptide can bind to the ligand binding site of the CD91 polypeptide fragment or CD91 endogenous and block the interaction of an HSP or HSP complex.

Such peptides may be produced synthetically or by using standard molecular biology techniques. Amino acid sequences and nucleotide sequences of naturally occurring CD91 ligands, such as HSPs, are generally available in sequence databases, such as GenBank. Computer programs, such as Entrez, can be used to browse the database, and retrieve any amino acid sequence and genetic sequence data of interest by accession number.

Additionally, compounds, such as those identified via techniques such as those described herein above, in Section 5.2, that are capable of modulating a CD91 polypeptide fragment-ligand interaction can be administered using standard techniques that are well known to those of skill in the art.

5.3.1 Target Autoimmune Diseases

Autoimmune diseases that can be treated by the methods, CD91 polypeptide fragments, or compounds identified by the methods of the present invention include, but are not limited to, insulin dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease. The diseases set forth above, as referred to herein, include those exhibited by animal models for such diseases, such as, for example non-obese diabetic (NOD) mice for IDDM and experimental autoimmune encephalomyelitis (EAE) mice for multiple sclerosis.

The methods and compounds identified by the methods of the present invention can be used to treat such autoimmune diseases by reducing or eliminating the immune response to the patient's own (self) tissue, or, alternatively, by reducing or eliminating a pre-existing autoimmune response directed at tissues or organs transplanted to replace self tissues or organs damaged by the autoimmune response.

5.3.2 Target Infectious Diseases

The infectious diseases that can be treated or prevented using the methods, CD91 polypeptide fragments, and compounds identified by the methods of the present invention include those caused by intracellular pathogens such as viruses, bacteria, protozoans, and intracellular parasites. Viruses include, but are not limited to viral diseases such as those caused by hepatitis type B virus, parvoviruses, such as adeno-associated virus and cytomegalovirus, papovaviruses such as papilloma virus, polyoma viruses, and SV40, adenoviruses, herpes viruses such as herpes simplex type I (HSV-I), herpes simplex type II (HSV-II), and Epstein-Barr virus, poxviruses, such as variola (smallpox) and vaccinia virus, RNA viruses, including but not limited to human immunodeficiency virus type I (HIV-I), human immunodeficiency virus type II (HIV-II), human T-cell lymphotropic virus type I (HTLV-I), and human T-cell lymphotropic virus type II (HTLV-II); influenza virus, measles virus, rabies virus, Sendai virus, picomaviruses such as poliomyelitis virus, coxsackieviruses, rhinoviruses, reoviruses, togaviruses such as rubella virus (German measles) and Semliki forest virus, arboviruses, and hepatitis type A virus.

In another embodiment, bacterial infections can be treated or prevented such as, but not limited to disorders caused by pathogenic bacteria including, but not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhiimurium, Salmonella typhii, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In another preferred embodiment, the methods can be used to treat or prevent infections caused by pathogenic protozoans such as, but not limited to, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, and Plasmodium malaria.

5.3.3 Target Proliferative Cell Disorders

With respect to specific proliferative and oncogenic disease associated with HSP-α2M receptor activity, the diseases that can be treated or prevented by the methods CD91 polypeptide fragments, or compounds identified by the methods of the present invention include, but are not limited to: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's acroglobulinemia, and heavy chain disease.

Diseases and disorders involving a deficiency in cell proliferation or in which cell proliferation is desired for treatment or prevention, and that can be treated or prevented by inhibiting the α2M receptor function, include but are not limited to degenerative disorders, growth deficiencies, hypoproliferative disorders, physical trauma, lesions, and wounds; for example, to promote wound healing, or to promote regeneration in degenerated, lesioned or injured tissues, etc.

5.3.4 Use of CD91 Polypeptide Fragment as Dominant Negative Mutants

In another embodiment of the invention, dominant negative mutants (“dominant negatives”) may be used therapeutically to block the immune response to an HSP-antigen complex, e.g., to treat an auto-immune disorder. In general, such dominant-negatives are mutants which, when expressed, interact with ligand (i.e., HSP-antigenic molecule complex), but lack one or more functions, i.e. endocytotic functions and/or signaling functions, of normal CD91. Such mutants interfere with the function of normal CD91 in the same cell or in a different cell, e.g. by titration of HSP-peptide complexes from the wild type receptor. Such a mutation, for example, can be one or more point mutation(s), a deletion, insertion, or other mutation in either the extracellular of the 515 kDa subunit, or the extracellular, transmembrane or intracellular domains of the 85 kDa subunit of the alpha(2) macroglobulin receptor (see Krieger and Herz, 1994, Annu. Rev. Biochem 63:601-637 for CD91 subunit configuration). However, in construction of dominant negative mutations in the either subunit, care should be taken to ensure that the cleavage domain (signaling cleavage between amino acids 3525 and 3526 of the precursor of CD91) remains intact so that the 515 kDa subunit is processed and presented on the cell surface. Additionally, care should be taken to ensure that the domains by which the two subunits associate should also remain functional. For example, in a specific embodiment, the C-terminal intracellular domain of the 85 kDa subunit is truncated. In another embodiment, a point mutation on the N-terminal 515 kDa subunit blocks endocytosis but not ligand binding. In another embodiment, the N-terminal 515 kDa subunit is expressed as a fusion protein, wherein the C-terminus of said fusion protein is the transmembrane domain and optionally the intracellular domain, of another Type 1 single transmembrane receptor.

Expression of a such a dominant negative mutation in cell can block uptake of ligand by normal functional receptors in the same or neighboring cells by titrating out the amount of available ligand. Thus, a recombinant antigen presenting cell expressing such a dominant negative can be used to titrate out HSP-antigenic molecule complexes when administered to a patient in need of treatment for an autoimmune disorder.

CD91 Polypeptide Fragments

In one embodiment of the present invention, an HSP-CD91 competitive antagonist is a CD91 polypeptide fragment, preferably a soluble peptide, that can bind to HSPs and therefore competitively inhibit or decrease HSP binding to the native (e.g. full length CD91) receptor.

Functional expression of HSP-binding portions of CD91 polypeptide fragment is preferably carried out as described for the CR8 domain by Huang et al., 1999, J. Biol. Chem 274:14130-14136. Briefly, to maintain proper folding, the protein is expressed as a GST fusion, expressed recombinantly, the GST portion cleaved, uncleaved protein removed on GSH-Sepharose, and cleaved protein refolded. Since the complement repeats bind to calcium, proper folding is assayed by measuring the binding of the refolded protein to calcium.

In a specific mode of the embodiment, an HSP-binding portion of a CD91 polypeptide fragment consists of or comprises at least one complement repeat, most preferably selected from CR3—CR10. In another specific mode of the embodiment, an HSP-binding portion of a CD91 polypeptide fragment comprises a cluster of complement repeats, most preferably C1-II. In other modes of the embodiment, the HSP-binding portion consists of at least 10, more preferably at least 20, yet more preferably at least 30, yet more preferably at least 40, and most preferably at least 80 (continuous) amino acids. In specific modes of the embodiment, such fragments are not larger than 40-45 amino acids. In other specific modes of the embodiment, such fragments are not larger than 80-90 amino acids.

Exemplary preferred peptides include but are not limited to those consisting of amino acids of SEQ ID NOs:2 or 3 of human p95.

Derivatives or analogs of HSP-binding portions of a CD91 polypeptide fragment are also contemplated as competitive antagonists of HSP-CD91 polypeptide fragment complexes. Such derivative or analogs include but are not limited to those molecules comprising regions that are substantially homologous to the extracellular domain of a CD91 polypeptide fragment or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a sequence encoding a CD91 polypeptide fragment that binds HSP, such hybridizing occurring under stringent, moderately stringent, or nonstringent conditions. In certain specific embodiments, a CD91 polypeptide fragment derivative is a chimeric or fusion protein comprising an HSP-binding portion of a CD91 polypeptide fragment, preferably consisting of at least one complement repeat of C1-II) joined at its amino- or carboxy-terminus via a peptide bond to an amino acid sequence of a different protein. Such a chimeric protein can be produced recombinantly as described above, by omitting the cleavage repurification steps.

Other HSP-binding CD91 polypeptide fragment derivatives can be made by altering CD91 polypeptide fragment coding sequences by substitutions, additions or deletions that provide for functionally equivalent molecules. Due to the degeneracy of nucleotide coding sequences, other DNA sequences which encode substantially the same amino acid sequence as an HSP-binding CD91 polypeptide fragment coding sequence or fragment thereof may be used in the practice of the present invention. Selection of suitable alterations and production of HSP-binding CD91 polypeptide fragment derivatives can be made applying the same principles described above for CD91 polypeptide fragment derivatives and using the general methods described in Sections 5.1.2 and 5.1.3.

HSP Peptides

In another mode of the embodiment, the antagonist is an peptide which comprises at least contiguous 10 amino acids of an HSP sequence. Such a peptide can bind to the ligand binding site of a CD91 polypeptide fragment and block the interaction of an HSP or HSP complex. Such peptides may be produced synthetically or by using standard molecular biology techniques. Amino acid sequences and nucleotide sequences of naturally occurring HSPs are generally available in sequence databases, such as GenBank. Computer programs, such as Entrez, can be used to browse the database, and retrieve any amino acid sequence and genetic sequence data of interest by accession number. Methods for recombinant and synthetic production of such peptides are described in Sections 5.1.2 and 5.1.3. Additionally, compounds, such as those identified via techniques such as those described herein above, in Section 5.2, that are capable of modulating a CD91 polypeptide fragment activity can be administered using standard techniques that are well known to those of skill in the art.

RAP

In one embodiment of the present invention, an HSP-CD91 polypeptide fragment competitive antagonist is α2MR-associated protein (RAP) (Genbank accession no. A39875) or a CD91 polypeptide fragment-binding portion thereof. In a specific mode of the embodiment, an CD91 polypeptide fragment-binding portion of RAP consists of or comprises a fragment of the RAP RBD consisting of at least 10 (continuous) amino acids. In other modes of the embodiment, the fragment consists of at least 20, 30, 40, 50, 75 or 100 amino acids of the RBD. In specific modes of the embodiment, such fragments are not larger than 28, 50 or 100 amino acids. In other specific modes of the embodiment, a p95 protein-binding portion of RAP comprises a CD91 polypeptide fragment-binding portion of domain 1 or 3, e.g. as depicted in Nielsen et al., supra, FIG. 3, Group D or E. Expression of recombinant RAP or a CD91 polypeptide fragment-binding portion thereof, is preferably achieved as described by Anderson, U.S. Pat. No. 5,399,349.

### 6. EXAMPLE

### Cloning, Expression, and Purification of p95 and p110

6.1 Introduction

The Example presented herein describes the successful identification of an interaction between gp96, HSP90, HSP70, and calreticulin with CD91 present in macrophages and dendritic cells. The experiments presented herein form the basis for the purification of a p95 or p110 polypeptide or a fragment or an analog, derivative or mimetic thereof and for the screening and therapeutic methods of the present invention.

6.2 Materials and Methods

Purification of HSPs. HSPs were purified as described (Srivastava, P. K., 1997, Methods: A companion to Methods in Enzymology 12:165-171; Basu and Srivastava, 1999, J. Exp. Med. 189(5):797-802). All buffers used for purifications were prepared with endotoxin free water (Nanopure Infinity UV/UF, Bamstead/Thermolyne, Dubuque, Iowa) and all glasswares used for purification were cleaned with endotoxin free water and baked in a 400iF oven (Gruenberg, Wlliamsport, Pa.). The HSP-containing fractions were identified by immunoblots.

Conjugation of proteins to FITC and staining of cells. Purified proteins were conjugated to FITC using the FluoroTag FITC conjugation kits (SIGMA) as per the manufacturers protocol. Conjugation was confirmed by a 2 kDa increase in molecular weight by SDS-PAGE and by inimunoblotting with an anti-FITC monoclonal antibody.

Incubations of indicated amounts of FITC-tagged proteins and cells were done in the presence of 1% nonfat dry milk (Carnation®) in PBS for 20 min at 4° C. After repeated washing, cells were analyzed by flow cytometry (Becton Dickenson, La Jolla, Calif.). Cells were also labeled with propidium iodide just before FACScan analysis. Cells staining positive for propidium iodide were gated out of the events. No differences were observed in the binding of HSPs to Mac-1+ cells from pristaned or non-pristaned mice. Fixed or unfixed cells were labeled with FITC-tagged HSP as above. Labeled cells were visualized using a Zeiss LSM confocal microscope.

Affinity chromatography. Proteins (1 mg) in 2 ml volume were incubated with 2 ml of equilibrated AminoLink beads in PBS with a reductant (NaCNBH₃) for 1 hour. Uncoupled protein was removed by extensive washing of the column and unreactive groups quenched. Immobilization yields were typically >92% of the starting amount of protein. Columns were stored at 4° C. until used. Such columns were made with antibodies described herein (purified as described in Srivastava et al., 1986, Proc. Natl. Acad. Sci., U.S.A. 83:3407-3411) and albumin.

Purification of Recombinant Human CD91-p95 or p110 fragment A construct was engineered to express and export soluble, epitope-tagged CD91-p95 or p110 protein. The construct comprised the p95 or p 110 cDNA fragment of human CD91 (SEQ ID NO:1 or 12 respectively) operably linked to synthetic oligonucleotides encoding a thrombin protease recognition and cleavage site, thrombin, monoconal antibody epitope, an HA(haemagglutinin)(12CA5) epitope, biotin-ligase recognition sequence (AviTag Biotin Ligase) and 6×-His hexa-histidine purification sequence just prior to an engineered stop codon. Expression plasmid DNA was transfected into HEK-293. After drug resistance selection, supernatants from cell clones secreting high levels of recombinant epitope-tagged human CD91-p95 or p 110 protein were applied to anti-HA affinity resins for single step purification of recombinant human CD91-p95 or p110 protein. ELISA assays were performed with HRP conjugated Anti-penta-His antibodies and anti-HA antibodies that bind to epitopes on tagged protein fragments to confirm presence of protein fragment. ELISA assays were performed with HRP conjugated anti-HA antibodies and anti-CD91 (8G1) antibodies that bind to epitopes on tagged protein fragment to confirm presence of protein fragment. After drug resistance selection, supernatants from cell clones secreting high levels of recombinant epitope-tagged human CD91-p95 or p110 protein can also be applied to Ni-NTA affinity resins for single step purification of recombinant human CD91-p95 or p110 protein.

6.3 Results

Expression of p80

Random decamers were used in an RT-PCR reaction to synthesize cDNA from total human liver RNA (April 2001). Synthetic oligonucleotide primers specific to a defined fragment of the CD91 gene (Human mRNA for LDL receptor related protein; GenBank Accession #X13916) amplified the expected 2.35 kB product from this cDNA. The amplicon was cloned and sequenced. Primers were chosen to amplify a region of this sequence encoding for the first 728 amino acids of CD91. The molecular weight of the translated protein from this 728 amino acid N-terminal portion of CD91 is approximately 80 kD.

A construct was engineered to express and export a soluble, epitope-tagged CD91-p80 protein. A “tag” sequence was added, in-frame, to the end of the CD91-p80 PCR product that encoded for thrombin, an HA(haemagglutinin)(12CA5) epitope, biotin-ligase recognition sequence (AviTag Biotin Ligase) and 6×-His hexa-histidine purification sequence just prior to an engineered stop codon. This construct was cloned into mammalian expression vector pCDNA3 (Invitrogen) and transfected into a variety of mammalian cell lines. After failing to detect any secreted recombinant CD91-p80 in transfection supernatants, an IFA was performed on transfected cells using anti-HA-FITC. The IFA clearly showed individual cells to brightly stain anti-HA-FITC. These cells non-productively express HA peptide linked to a CD91 fragment. However, this recombinant CD91-p80, presumably mis-folded and sequestered in the endoplasmic reticulum.

Thus, the p80 construct engineered to express and export soluble, epitope-tagged CD91-p80 expressed protein, only produced a protein which was retained in intracellular compartments.

Expression of p95 and p110

Applicants recognized the importance of the CD91 tertiary structure (most notably the six YWTD beta-propeller motifs) and its effect on expression of secreted, functional recombinant protein. The CD91-p80 construct terminates in a position which disrupts a beta-propeller motif. An extension to the CD91-p80 clone was designed to alleviate this problem. The extension was designed to encode an additional 123 amino acids of CD91 resulting in the cDNA encoding p95, ending in a region that preserves the well-defined tertiary structure. With the added extension, the molecular weight of the translated recombinant protein is approximately 95 kD (851 amino acids from the N-terminus of CD91). The resulting CD91 fragment was named p95 (SEQ ID NO:2).

For p110, an additional 135 amino acids of CD91 were added. With the added extension, the molecular weight of the translated recombinant protein is approximately 110 kD (986 amino acids from the N-terminus of CD91), and the resulting CD91 fragment was named p 110 (SEQ ID NO:12 and 13).

The p95 extension was generated by PCR using a 3.3 kB cloned region of the 5′-end of human CD91 RNA as a template. The extension was cloned, in-frame, into the existing CD91-p80 clone and maintains all the C-terminal “tag” sequences.

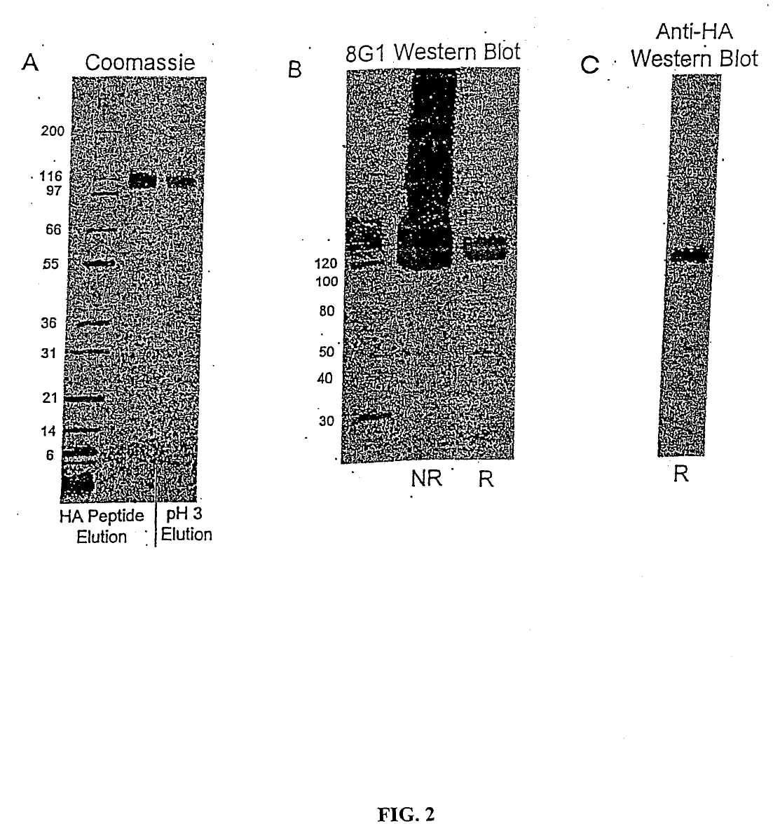

A soluble, epitope-tagged CD91-p95 protein was successfully expressed and exported using the construct. The expressed protein consisted of the p95 fragment, Thrombin, HA epitope (12CA5), AviTag Biotin Ligase, and 6×-His, (SEQ ID NO:3). Cells transfected with this construct cloned into pcDNA3 (Invitrogen) and pcDNA5/FRT/TO (Invitrogen) expression vectors have been shown by ELISA to secrete a protein in the 100 kD range that is recognized by anti-HA and anti-CD91 (8G1). The p95 epitope-tagged protein eluted from the HA affinity column was visualized by staining with Coomassie Brilliant Blue (FIG. 2). The expressed protein was not restricted to intracellular compartments as was the case with the p80 fragment of CD91.

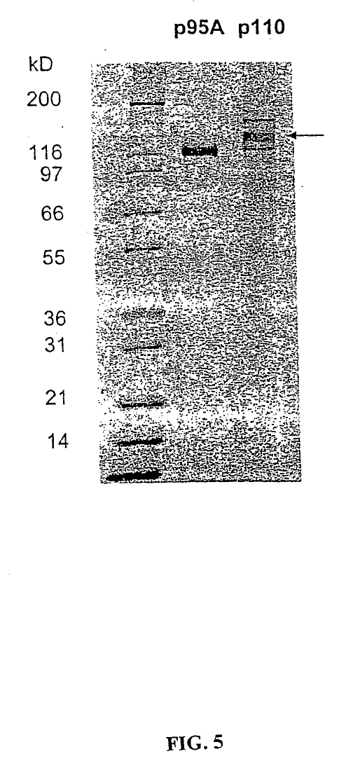

Similarly, the p110 expression construct was constructed and the expressed protein purified by affinity chromatography using Ni-nitrolotiacetic (NTA) (FIG. 5).

These methods are also used to recombinantly express CD91 polypeptide fragments of the invention that comprise CD91 polypeptides comprising amino acid sequence extending into domain II region of CD91. In other embodiments, the CD91 fragment comprises p95 plus additional amino acid sequences extending into domain III region of CD91. In yet other embodiments, the CD91 fragment comprises additional amino acid sequences extending into domain IV region of CD91. In yet other embodiments, the CD91 fragment comprises fragments of specific amino acid residues as described in Section 5.2 above.

Expression of p282

The p282 CD91 fragment of cDNA encodes four additional YWTD beta propeller structural motifs, which extends to amino acid residue 2517 of the CD91 sequence SEQ ID NO:2 (see FIG. 4 (SEQ ID NO:7)). In particular, the p282 fragment comprises amino acid residues that encode four additional propeller regions than p110 corresponding to amino acid residues 1269-1531, 1583-1846, 1934-2151, and 2199-2473, respectively, of SEQ ID NO: 2. Extensions to additional amino acids preserves the well-defined tertiary structure necessary for recombinant expression. With the added extension, the molecular weight of the translated recombinant protein is approximately 282 kD (2517 amino acids from the N-terminus of CD91). The resulting CD91 fragment is named p282 (SEQ ID NO:7).

The p282 extension is generated by PCR using a cloned region of the human CD91 RNA as a template. The extension is cloned, in-frame, into the existing CD91-p80 clone and maintains all the C-terminal “tag” sequences.

A soluble, epitope-tagged CD91-p282 protein is successfully expressed and exported using the construct. The expressed protein consists of the p282 fragment, Thrombin, HA epitope (12CA5), AviTag Biotin Ligase, and 6×-His, (SEQ ID NO:6). Cells transfected with this construct cloned into pcDNA3 (Invitrogen) and pcDNA5/FRT/TO (Invitrogen) expression vectors are confirmed by ELISA to secrete a protein in the 290 kD range that is recognized by anti-HA and anti-CD91. The p282 epitope-tagged protein eluted from the HA affinity column is visualized by staining with Coomassie Brilliant Blue. The expressed protein is not restricted to intracellular compartments as is the case with the p80 fragment of CD91. The expressed p282 protein is purified by affinity chromatography using Ni-nitrolotiacetic (NTA).

Expression of p373

The p373 CD91 fragment of cDNA encodes a complete additional YWTD beta propeller structural motif, which extends to amino acid residue 3330 of the CD91 sequence SEQ ID NO:2 (see FIG. 4 (SEQ ID NO:9)). In particular, the p373 fragment comprises amino acid residues that encode one additional propeller region than p282, corresponding to amino acid residues 3019-3284 of SEQ ID NO:2. Extensions to additional amino acids preserves the well-defined tertiary structure necessary for recombinant expression. With the added extension, the molecular weight of the translated recombinant protein is approximately 373 kD (3330 amino acids from the N-terminus of CD91). The resulting CD91 fragment is named p373 (SEQ ID NO:9).

The p373 extension is generated by PCR using a 3.3 kB extension is generated by PCR using a cloned region of the human CD91 RNA as a template. The extension is cloned, in-frame, into the existing CD91-p80 clone and maintains all the C-terminal “tag” sequences.

A soluble, epitope-tagged CD91-p373 protein is successfully expressed and exported using the construct. The expressed protein consists of the p373 fragment, Thrombin, HA epitope (12CA5), AviTag Biotin Ligase, and 6×-His, (SEQ ID NO:8). Cells transfected with this construct cloned into pcDNA3 (Invitrogen) and pcDNA5/FRT/TO (Invitrogen) expression vectors are confirmed by ELISA to secrete a protein in the 380 kD range that is recognized by anti-HA and anti-CD91. The p373 epitope-tagged protein eluted from the HA affinity column is visualized by staining with Coomassie Brilliant Blue. The expressed protein is not restricted to intracellular compartments as is the case with the p80 fragment of CD91. The expressed p373 protein is purified by affinity chromatography using Ni-nitrolotiacetic (NTA).

Expression of p494

The p494 CD91 fragment of cDNA encodes an additional YWTD beta propeller structural motifs, which extends to amino acid residue 4420 of the CD91 sequence SEQ ID NO:2 (see FIG. 4 (SEQ ID NO:11)). In particular, the p494 fragment comprises amino acid residues that encode one additional propeller region than p373, corresponding to amino acid residues 3828-4142 of SEQ ID NO:2. The p494 fragment also comprises amino acid residues that encode a furin cleavage site at residue 3943 of SEQ ID NO:2 and CHO side groups at residues 4075 and 4125 of SEQ ID NO:2. Applicants recognized the importance of the CD91 tertiary structure encoded by p95 (most notably the six YWTD beta-propeller motifs) and its effect on expression of secreted, functional recombinant protein. Extensions to additional amino acids preserves the well-defined tertiary structure necessary for recombinant expression. With the added extension, the molecular weight of the translated recombinant protein is approximately 494 kD (4420 amino acids from the N-terminus of CD91). The resulting CD91 fragment is named p494 (SEQ ID NO:11).

The p494 extension is generated by PCR using a 3.3 kB extension is generated by PCR using a cloned region of the human CD91 RNA as a template. The extension is cloned, in-frame, into the existing CD91-p80 clone and maintains all the C-terminal “tag” sequences.

A soluble, epitope-tagged CD91-p494 protein is successfully expressed and exported using the construct. The expressed protein consists of the p494 fragment, Thrombin, HA epitope (12CA5), AviTag Biotin Ligase, and 6×-His, (SEQ ID NO:10). Cells transfected with this construct cloned into pcDNA3 (Invitrogen) and pcDNA5/FRT/TO (Invitrogen) expression vectors are confirmed by ELISA to secrete a protein in the 500 kD range that is recognized by anti-HA and anti-CD91. The p494 epitope-tagged protein eluted from the HA affinity column is visualized by staining with Coomassie Brilliant Blue. The expressed protein is not restricted to intracellular compartments as is the case with the p80 fragment of CD91. The expressed p494 protein is purified by affinity chromatography using Ni-nitrolotiacetic (NTA).

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

What is claimed:

1. A nucleic acid molecule comprising the nucleotide sequence of SEQ ID NO:1, wherein the nucleotide sequence is not flanked by sequences adjacent to SEQ ID NO:1 in the native CD91 nucleotide sequence.

2. A nucleic acid molecule comprising a nucleotide sequence which encodes a polypeptide consisting of the amino acid sequence of SEQ ID NO:2, 3, 6, 7, 8, 9, 10, 11, or 12.

3. A nucleic acid molecule comprising a nucleotide sequence which 1) hybridizes over its full length to the nucleotide sequence of SEQ ID NO:1 under conditions of stringent washing and 2) encodes a polypeptide that is capable of (i) being recombinantly produced and secreted into culture medium and (ii) binding to a heat shock protein.

4. A nucleic acid molecule comprising a nucleotide sequence which 1) hybridizes over its full length to the nucleic acid sequence of SEQ ID NO: 1, 14, 15, 16, or 17 under conditions of stringent washing; and 2) encodes a polypeptide that is capable of (i) being recombinantly produced and secreted into culture medium and (ii) binding to a heat shock protein.

5. A nucleic acid molecule comprising a nucleotide sequence which encodes a polypeptide comprising the amino acid sequence of SEQ ID NO: 2, 3, 6, 7, 8, 9, 10, 11, or 12, wherein said polypeptide interacts with a heat shock protein.

6. The isolated nucleic acid molecule of any one of claims 1-5, further comprising a heterologous nucleotide sequence.

7. A vector comprising the nucleic acid molecule of any one of claims 1-5.

8. An expression vector comprising the nucleic acid molecule of any one of claims 1-5, wherein the nucleotide sequence is operatively associated with a nucleotide regulatory sequence that controls expression of the nucleotide sequence in a host cell.

9. A genetically engineered host cell comprising the nucleic acid molecule of any one of claims 1-5, wherein the nucleotide sequence is operatively associated with a nucleotide regulatory sequence that controls expression of the nucleotide sequence in the host cell.

10. A host cell comprising the expression vector of claim 8.

11. A polypeptide comprising the amino acid sequence of SEQ ID NO:2, 3, 6, 7, 8, 9, 10, 11, or 12, wherein the amino acid sequence is not flanked by sequences adjacent to SEQ ID NO:2, 3, 6, 7, 8, 9, 10, 11, or 12, respectively, in the native CD91 polypeptide sequence.

12. An isolated polypeptide comprising a contiguous amino acid sequence, wherein said amino acid sequence consists of amino acid residues 1-851 of SEQ ID NO:11, fused to one or more contiguous amino acids of amino acid residues 852-4420 of SEQ ID NO:11.

13. An isolated polypeptide encoded by a nucleic acid molecule that hybridizes under stringent conditions to a complement of SEQ ID NO: 1, 14, 15, 16, or 17, wherein said polypeptide binds to a heat shock protein.

14. An isolated polypeptide encoded by a nucleic acid molecule which hybridizes under stringent conditions to a complement of a nucleic acid molecule consisting of a nucleotide sequence that encodes a polypeptide consisting of the amino acid sequence of SEQ ID NO:2, 6, 8, 10, 11, or 12, wherein said polypeptide binds to a heat shock protein.

15. The polypeptide of claim 13 or 14, wherein the heat shock protein is selected from the group consisting of a gp96, hsp 90, hsp 70, and calreticulin.

16. The polypeptide of claim 15, wherein the heat shock protein is gp96.

17. A fusion polypeptide comprising the amino acid sequence of SEQ ID NO:2, 3, 6, 7, 8, 9, 10, 11, or 12 and a heterologous amino acid sequence.

18. A method for making the polypeptide of any one of claims 12-17, comprising the steps of:
(a) culturing a cell comprising a recombinant nucleotide sequence encoding the polypeptide of any one of claims 11-17, under conditions such that the polypeptide is expressed by said cell; and
(b) recovering the expressed polypeptide from the cell culture.

19. An antibody or fragment thereof that immunospecifically binds to a CD91 polypeptide fragment, wherein the CD91 polypeptide fragment comprises the amino acid sequence of SEQ ID NO.:2, 3, 6, 7, 8, 9, 10, 11, or 12.

20. The antibody or fragment of claim 19, wherein the antibody is a polyclonal antibody, a monoclonal antibody, a humanized antibody, a single chain antibody, or a chimeric antibody.

21. The antibody or fragment of claim 20, wherein the antibody or fragment thereof is a Fab fragment.

22. An anti-idiotype antibody which binds to the antibody or fragment of claim 19.

23. A kit comprising the antibody of claim 19 and instructions for use of the antibody to detect a CD91 polypeptide fragment.

24. A method for treating a CD91-related disease or disorder comprising administering the polypeptide of any one of claims 11-17 to a mammal in need thereof in an amount effective to treat the disease or disorder.

25. The method of claim 24, wherein the disease or disorder is an autoimmune disorder, a disease or disorder involving disruption of antigen presentation or endocytosis, a disease or disorder involving cytokine clearance or inflammation, a proliferative disorder, a viral disorder or other infectious disease, hypercholesterolemia, Alzheimer's disease, diabetes, or osteoporosis.

26. A method for identifying a compound that modulates an HSP-CD91-mediated process, comprising:
(a) contacting a test compound with a heat shock protein and a CD91 polypeptide fragment that binds a CD91 ligand; and
(b) measuring the level of the CD91 polypeptide fragment activity or expression,
such that if the level of activity or expression measured in (b) differs from the level of the CD91 polypeptide fragment activity or expression measured in the presence the heat shock protein but in the absence of the test compound, then a compound that modulates an HSP-CD91-mediated process is identified.

27. The method of claim 26, wherein the compound identified is an antagonist which interferes with the interaction of the heat shock protein with the CD91 polypeptide fragment.

28. The method of claim 26 in which the test compound is a small molecule or a peptide.

29. The method of claim 28 in which the peptide comprises at least 5 consecutive amino acids of the CD91 polypeptide fragment.

30. The method of claim 26 in which the HSP-CD91-mediated process affects an autoimmune disorder, a disease or disorder involving disruption of antigen presentation or endocytosis, a disease or disorder involving cytokine clearance or inflammation, a proliferative disorder, a viral disorder or other infectious disease, hypercholesterolemia, Alzheimer's disease, diabetes, or osteoporosis.

31. A method for identifying a compound that modulates the binding of a heat shock protein to CD91, comprising:
(a) contacting a heat shock protein with a CD91 polypeptide fragment, or derivative thereof, which CD91 polypeptide fragment binds a CD91 ligand, in the presence of a test compound under conditions conducive to binding; and
(b) measuring the level of heat shock protein bound to the CD91 polypeptide fragment, analog, derivative or mimetic thereof,
such that if the level of bound heat shock protein measured in (b) differs from the level of heat shock protein measured bound to the CD91 polypeptide fragment or analog, derivative, or mimetic thereof measured under said conditions in the absence of the test compound, then a compound that modulates the binding of an HSP to the CD91 polypeptide fragment is identified.

32. The method of claim 31 wherein the CD91 polypeptide fragment, or derivative thereof, is immobilized to a solid support.

33. The method of claim 32 wherein the solid support is a microtiter dish.

34. The method of claim 31 wherein the level of bound heat shock protein is measured using a heat shock protein-specific antibody.

35. The method of claim 31 wherein the heat shock protein is labeled and the level of bound heat shock protein is measured by detecting the label.

36. The method of claim 35 wherein the heat shock protein is labeled with a fluorescent label.


SEQUENCE LISTING (17 sequences)

SEQ ID NO: 1 - DNA, 2553 bp, H. sapiens
atgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctcca

SEQ ID NO: 2 - protein, 851 aa, H. sapiens
MLTPPLLLLLPLLSALAAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIMVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPP

SEQ ID NO: 3 - protein, 896 aa, H. sapiens
MLTPPLLLLLPLLSALAAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIMVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPGGRLVPRGSYPYDVPDYAIEGHVGLNDIFEAQKIEWHEGHHHHHH

SEQ ID NO: 4 - protein, 844 aa, M.
MLTPPLLLLVPLLSALVSGATMDAPKTCSPKQFACRDQITCISKGWRCDGERDCPDGSDEAPEICPQSKAQRCPPNEHSCLGTELCVPMSRLCNGIQDCMDGSDEGAHCRELRANCSRMGCQHHCVPTPSGPTCYCNSSFQLEADGKTCKDFDECSVYGTCSQLCTNTDGSFTCGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANTQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVAGAPPTVTLLRSERPPIFEIRMYDAHEQQVGTNKCRVNNGGCSSLCLATPGSRQCACAEDQVLDTDGVTCL

SEQ ID NO: 5 - protein, 4544 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 6 - protein, 2565 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVGGRLVPRGSYPYDVPDYAIEGHVGLNDIFEAQKIEWHEGHHHHHH

SEQ ID NO: 7 - protein, 2520 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAV

SEQ ID NO: 8 - protein, 4464 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGGGRLVPRGSYPYDVPDYAIEGHVGLNDIFEAQKIEWHEGHHHHHH

SEQ ID NO: 9 - protein, 4419 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPG

SEQ ID NO: 10 - protein, 4464 aa, H. sapiens; identical to SEQ ID NO: 8

SEQ ID NO: 11 - protein, 4419 aa, H. sapiens; identical to SEQ ID NO: 9

SEQ ID NO: 12 - protein, 986 aa, H. sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNN

SEQ ID NO: 13 - protein, 896 aa, H. sapiens; identical to SEQ ID NO: 3

SEQ ID NO: 14 - protein, 2688 aa, H. sapiens
ATGCTGACCCCGCCGTTGCTCCTGCTGCTGCCCCTGCTCTCAGCTCTGGTCGCGGCGGCTATCGACGCCCCTAAGACTTGCAGCCCCAAGCAGTTTGCCTGCAGAGATCAAATAACCTGTATCTCAAAGGGCTGGCGGTGCGACGGTGAGAGGGACTGCCCAGACGGATCTGACGAGGCCCCTGAGATTTGTCCACAGAGTAAGGCCCAGCGATGCCAGCCAAACGAGCATAACTGCCTGGGTACTGAGCTGTGTGTTCCCATGTCCCGCCTCTGCAATGGGGTCCAGGACTGCATGGACGGCTCAGATGAGGGGCCCCACTGCCGAGAGCTCCAAGGCAACTGCTCTCGCCTGGGCTGCCAGCACCATTGTGTCCCCACACTCGATGGGCCCACCTGCTACTGCAACAGCAGCTTTCAGCTTCAGGCAGATGGCAAGACCTGCAAAGATTTTGATGAGTGCTCAGTGTACGGCACCTGCAGCCAGCTATGCACCAACACAGACGGCTCCTTCATATGTGGCTGTGTTGAAGGATACCTCCTGCAGCCGGATAACCGCTCCTGCAAGGCCAAGAACGAGCCAGTAGACCGGCCCCCTGTGCTGTTGATAGCCAACTCCCAGAACATCTTGGCCACGTACCTGAGTGGGGCCCAGGTGTCTACCATCACACCTACGAGCACGCGGCAGACCACAGCCATGGACTTCAGCTATGCCAACGAGACCGTATGCTGGGTGCATGTTGGGGACAGTGCTGCTCAGACGCAGCTCAAGTGTGCCCGCATGCCTGGCCTAAAGGGCTTCGTGGATGAGCACACCATCAACATCTCCCTCAGTCTGCACCACGTGGAACAGATGGCCATCGACTGGCTGACAGGCAACTTCTACTTTGTGGATGACATCGATGATAGGATCTTTGTCTGCAACAGAAATGGGGACACATGTGTCACATTGCTAGACCTGGAACTCTACAACCCCAAGGGCATTGCCCTGGACCCTGCCATGGGGAAGGTGTTTTTCACTGACTATGGGCAGATCCCAAAGGTGGAACGCTGTGACATGGATGGGCAGAACCGCACCAAGCTCGTCGACAGCAAGATTGTGTTTCCTCATGGCATCACGCTGGACCTGGTCAGCCGCCTTGTCTACTGGGCAGATGCCTATCTGGACTATATTGAAGTGGTGGACTATGAGGGCAAGGGCCGCCAGACCATCATCCAGGGCATCCTGATTGAGCACCTGTACGGCCTGACTGTGTTTGAGAATTATCTCTATGCCACCAACTCGGACAATGCCAATGCCCAGCAGAAGACGAGTGTGATCCGTGTGAACCGCTTTAACAGCACCGAGTACCAGGTTGTCACCCGGGTGGACAAGGGTGGTGCCCTCCACATCTACCACCAGAGGCGTCAGCCCCGAGTGAGGAGCCATGCCTGTGAAAACGACCAGTATGGGAAGCCGGGTGGCTGCTCTGACATCTGCCTGCTGGCCAACAGCCACAAGGCGCGGACCTGCCGCTGCCGTTCCGGCTTCAGCCTGGGCAGTGACGGGAAGTCATGCAAGAAGCCGGAGCATGAGCTGTTCCTCGTGTATGGCAAGGGCCGGCCAGGCATCATCCGGGGCATGGATATGGGGGCCAAGGTCCCGGATGAGCACATGATCCCCATTGAAAACCTCATGAACCCCCGAGCCCTGGACTTCCACGCTGAGACCGGCTTCATCTACTTTGCCGACACCACCAGCTACCTCATTGGCCGCCAGAAGATTGATGGCACTGAGCGGGAGACCATCCTGAAGGACGGCATCCACAATGTGGAGGGTGTGGCCGTGGACTGGATGGGAGACAATCTGTACTGGACGGACGATGGGCCCAAAAAGACAATCAGCGTGGCCAGGCTGGAGAAAGCTGCTCAGACCCGCAAGACTTTAATCGAGGGCAAAATGACACACCCCAGGGCTATTGTGGTGGATCCACTCAATGGGTGGATGTACTGGACAGACTGGGAGGAGGACCCCAAGGACAGTCGGCGTGGGCGGCTGGAGAGGGCGTGGATGGATGGCTCACACCGAGACATCTTTGTCACCTCCAAGACAGTGCTTTGGCCCAATGGGCTAAGCCTGGACATCCCGGCTGGGCGCCTCTACTGGGTGGATGCCTTCTACGACCGCATCGAGACGATACTGCTCAATGGCACAGACCGGAAGATTGTGTATGAAGGTCCTGAGCTGAACCACGCCTTTGGCCTGTGTCACCATGGCAACTACCTCTTCTGGACTGAGTATCGGAGTGGCAGTGTCTACCGCTTGGAACGGGGTGTAGGAGGCGCACCCCCCACTGTGACCCTTCTGCGCAGTGAGCGGCCCCCCATCTTTGAGATCCGAATGTATGATGCCCAGCAGCAGCAAGTTGGCACCAACAAATGCCGGGTGAACAATGGCGGCTGCAGCAGCCTGTGCTTGGCCACCCCTGGGAGCCGCCAGTGCGCCTGTGCTGAGGACCAGGTGTTGGACGCAGACGGCGTCACTTGCTTGGCGAACCCATCCTACGTGCCTCCACCCCAGTGCCAGCCAGGCGAGTTTGCCTGTGCCAACAGCCGCTGCATCCAGGAGCGCTGGAAGTGTGACGGAGACAACGATTGCCTGGACAACAGTGATGAGGCCCCAGCCCTCTGCCATCAGCACACCTGCCCC

SEQ ID NO: 15 - DNA, 7551 bp, H. sapiens
atgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgc

SEQ ID NO: 16 - DNA, 9900 bp, H. sapiens
atgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagcccagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggatgacggccggacgtgtgctgatgtggacgagtgcagcaccaccttcccctgcagccagcgctgcatcaacacccatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggc

SEQ ID NO: 17 - DNA, 13260 bp, H. sapiens
atgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagcccagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggatgacggccggacgtgtgctgatgtggacgagtgcagcaccaccttcccctgcagccagcgctgcatcaacacccatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggctgcagcaacctgtgcctgctgtcccccgggggagggcacaaatgtgcctgccccaccaacttctacctgggcagcgatgggcgcacctgtgtgtccaactgcacggctagccagtttgtatgcaagaacgacaagtgcatccccttctggtggaagtgtgacaccgaggacgactgcggggaccactcagacgagcccccggactgccctgagttcaagtgccggcccggacagttccagtgctccacaggtatctgcacaaaccctgccttcatctgcgatggcgacaatgactgccaggacaacagtgacgaggccaactgtgacatccacgtctgcttgcccagtcagttcaaatgcaccaacaccaaccgctgtattcccggcatcttccgctgcaatgggcaggacaactgcggagatggggaggatgagagggactgccccgaggtgacctgcgcccccaaccagttccagtgctccattaccaaacggtgcatcccccgggtctgggtctgcgaccgggacaatgactgtgtggatggcagtgatgagcccgccaactgcacccagatgacctgtggtgtggacgagttccgctgcaaggattcgggccgctgcatcccagcgcgttggaagtgtgacggagaggatgactgtggggatggctcggatgagcccaaggaagagtgtgatgaacgcacctgtgagccataccagttccgctgcaagaacaaccgctgcgtgcccggccgctggcagtgcgactacgacaacgattgcggtgacaactccgatgaagagagctgcacccctcggccctgctccgagagtgagttctcctgtgccaacggccgctgcatcgcggggcgctggaaatgcgatggagaccacgactgcgcggacggctcggacgagaaagactgcaccccccgctgtgacatggaccagttccagtgcaagagcggccactgcatccccctgcgctggcgctgtgacgcagacgccgactgcatggacggcagcgacgaggaggcctgcggcactggcgtgcggacctgccccctggacgagttccagtgcaacaacaccttgtgcaagccgctggcctggaagtgcgatggcgaggatgactgtggggacaactcagatgagaaccccgaggagtgtgcccggttcgtgtgccctcccaaccggcccttccgttgcaagaatgaccgcgtctgtctgtggatcgggcgccaatgcgatggcacggacaactgtggggatgggactgatgaagaggactgtgagccccccacagcccacaccacccactgcaaagacaagaaggagtttctgtgccggaaccagcgctgcctctcctcctccctgcgctgcaacatgttcgatgactgcggggacggctctgacgaggaggactgcagcatcgaccccaagctgaccagctgcgccaccaatgccagcatctgtggggacgaggcacgctgcgtgcgcaccgagaaagcggcctactgtgcctgccgctcgggcttccacaccgtgcccggccagcccggatgccaagacatcaacgagtgcctgcgcttcggcacctgctcccagctctgcaacaacaccaagggcggccacctctgcagctgcgctcggaacttcatgaagacgcacaacacctgcaaggccgaaggctctgagtaccaggtcctgtacatcgctgatgacaatgagatccgcagcctgttccccggccacccccattcggcttacgagcaggcattccagggtgacgagagtgtccgcattgatgctatggatgtccatgtcaaggctggccgtgtctattggaccaactggcacacgggcaccatctcctaccgcagcctgccacctgctgcgcctcctaccacttccaaccgccaccggcgacagattgaccggggtgtcacccacctcaacatttcagggctgaagatgcccagaggcatcgccatcgactgggtggccggaaacgtgtactggaccgactcgggccgagatgtgattgaggtggcgcagatgaagggcgagaaccgcaagacgctcatctcgggcatgattgacgagccccacgccattgtggtggacccactgagggggaccatgtactggtcagactggggcaaccaccccaagattgagacggcagcgatggatgggacgcttcgggagacactggtgcaggacaacattcagtggcccacaggcctggccgtggattatcacaatgagcggctgtactgggcagacgccaagctttcagtcatcggcagcatccggctcaatggcacggaccccattgtggctgctgacagcaaacgaggcctaagtcaccccttcagcatcgacgtctttgaggattacatctatggtgtcacctacatcaataatcgtgtcttcaagatccataagtttggccacagccccttggtcaacctgacagggggcctgagccacgcctctgacgtggtcctttaccatcagcacaagcagcccgaagtgaccaacccatgtgaccgcaagaaatgcgagtggctctgcctgctgagccccagtgggcctgtctgcacctgtcccaatgggaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacat